(19) United States
(12) Patent Application Publication
(10) Pub. No.: US 2023/0227901 A1
(43) Pub. Date: Jul. 20, 2023

(54) Selective Addition of Reagents to Droplets

(71) Applicant: The Regents of the University of California, Oakland, CA (US)

(72) Inventors: Adam R. Abate, Daly City, CA (US); Iain Clark, San Francisco, CA (US); Peng Xu, Redwood City, CA (US); Cyrus Modavi, San Francisco, CA (US)

(21) Appl. No.: 18/010,188

(22) PCT Filed: Jun. 24, 2021
(86) PCT No.: PCT/US2021/038937

§ 371 Date: Dec. 13, 2022

Related U.S. Application Data
(60) Provisional application No. 63/044,504, filed on Jun. 26, 2020.

(51) Int. Cl. C12Q 1/6869; B01L 3/00

(52) U.S. Cl. CPC C12Q 1/6869; B01L 3/502792; C12Q 2600/156; C12Q 2600/16; B01L 2300/0867; B01L 2300/0645; B01L 2300/0654; B01L 2200/0652; B01L 2400/0622; B01L 2200/16; B01L 2400/0427

(57) ABSTRACT

Methods for selectively adding one or more reagents are provided. In certain aspects, the methods include selectively merging one or more droplets of a plurality of droplets with one or more droplets of a plurality of reagent droplets based on detection of a property. Systems, devices and kits for practicing the subject methods are also provided. The subject disclosure may find use in a wide variety of applications, such as increasing the accuracy and/or efficiency of single-cell sequencing, detection of cancer or other diseases, monitoring disease progression, analyzing the DNA or RNA content of cells, and other applications in which it is desired to detect and/or quantify specific target cells.

### CROSS-REFERENCE

This application claims priority to U.S. Provisional Applications 63/044,504 filed Jun. 26, 2020, which is incorporated herein by reference.

### STATEMENT OF GOVERNMENT SUPPORT

This invention was made with government support under grant number NIAID U01A1129206 awarded by the National Institutes of Health (NIH). The government has certain rights in the invention.

### INTRODUCTION

A recent, high-value product of droplet microfluidics has been scalable and cost-effective single cell sequencing. This approach encapsulates individual cells in droplets with barcodes that uniquely label the genome (1, 2), transcriptome (3, 4), or proteome (5, 6). After barcoding, all material can be pooled, efficiently read by DNA sequencing, and separated in silico. Droplet microfluidic barcoding provides the throughput and precision necessary to characterize thousands of single cells and understand trajectories during cellular differentiation (7), heterogeneity in disease (8, 9), transcriptional changes associated with genetic perturbations (10, 11), as well as numerous other biological measurements. Indeed, the approach has heralded a new era in systems biology and enabled myriad systems to be decomposed into their most essential component, the single cell.

Existing technologies for high throughput droplet-based single cell sequencing analyze all cells in a sample. Often, however, cells of interest comprise a rare subset in a mixed population, necessitating sorting prior to barcoding. In addition to adding cumbersome steps, pre-sorting can harm cells and perturb gene expression. Moreover, if the target is rare, single cell analysis with droplet-based approaches may not be possible, because sorting doesn't yield enough cells to run existing workflows, which minimally require tens of thousands of input cells. One solution would be to integrate sorting directly into the barcoding chip, streamlining workflows for the user and reducing perturbations on cell gene expression, since barcoding can be performed within seconds of isolation. However, upgrading existing instruments to add sorting capability would require expensive and complex additions, including high-speed, high voltage electronics or acoustic deflectors. Moreover, droplet sorting (12-16) is one of the most challenging and user-input intensive operations in droplet microfluidics, constituting significant barriers to implementing it into commercial barcoding instruments that have been designed for simplicity and engineering reliability. To enable integration of subset analysis into single cell microfluidic workflows, a new paradigm is needed.

### SUMMARY

Described herein are methods to target subsets of analytes without the need for physical separation. This approach exploits the ability to selectively add reagents to droplets, perform reactions in those droplets, and thereby target subsets of droplets and their contents for subsequent analysis.

Selective addition of fluids to droplets enables reactions to occur only in droplets of interest. Chemicals, small molecules, proteins/peptides, deoxyribonucleic acid (DNA), ribonucleic acid (RNA), oligonucleotides, buffers, enzymes, beads, cells, or virus can be selectively added to droplets of interest so that reactions (synthesis, polymerase chain reaction (PCR), multiple displacement amplification (MDA), reverse transcription, transposase mediated genomic insertions, transfection, transduction, transformation, etc.) occur only in those droplets.

Described herein are methods to target droplet subpopulations, for example cell subsets contained in droplets, for analysis without the need for physical separation. This approach exploits the ability to selectively coalesce target droplets with essential reagents. Coalescence combines all components needed to catalyze the desired reaction, yielding products that can be recovered. By contrast, negative droplets are not coalesced, yielding no reaction products. In this way, selective coalescence achieves subset analysis without the need to pre-separate the input sample. The power of selective merger has been demonstrated by applying it to single cell RNA-sequencing and DNA sequencing of cancer hotspots for specific cells in a mixed population. The instant method comprises a simple and flexible strategy to integrate subset analysis into droplet-based single cell workflows.

Provided herein are methods of selectively adding one or more reagents to one or more target molecules. In some embodiments, the methods include: flowing an emulsion comprising a plurality of droplets through a microfluidic device, wherein at least one droplet of the plurality of droplets comprises a target molecule; flowing a plurality of reagent droplets comprising one or more reagents through the microfluidic device; detecting via an optical detector a property of one or more droplets of the plurality of droplets; and selectively merging one or more droplets of the plurality of droplets with one or more droplets of the plurality of reagent droplets based on the detection of the property.

The property can be an optical property. In some cases, the single cell is labelled with a first fluorescent moiety and the optical property is fluorescence of the first fluorescent moiety. In other cases the optical property is absorbance. In other cases the property is size. In other cases the property is conductivity, e.g. electrical conductivity. These properties can also be applied to each of the methods and systems described below.

The selective merging can be performed in any suitable manner. For example, an electric field, stream merging, pico-injection, or triple-emulsion coalescence can be used. In some cases the selective merging comprises applying an electric field to selectively merge the one or more droplets with the one or more droplets of the plurality of reagent droplets. In other cases the selective merging comprises merging the one or more droplets with one or more droplets of the plurality of reagent droplets. In other cases the selective merging comprises pico-injection. In other cases the selective merging comprises triple-emulsion coalescence. These manners of selective merging can also be applied to each of the methods and systems described below.

Also described herein are methods of single-cell sequencing comprising selectively adding one or more reagents to one or more target cells. In certain embodiments, the methods comprise: flowing an emulsion comprising a plurality of droplets through a microfluidic device, wherein at least one droplet of the plurality of droplets comprises a single cell; flowing a plurality of reagent droplets comprising one or more reagents through the microfluidic device; detecting via a detector a property of one or more droplets of the plurality of droplets; selectively merging one or more droplets of the plurality of droplets with one or more droplets of the plurality of reagent droplets based on the property; and sequencing the selectively merged one or more droplets of the plurality of droplets.

In some cases, a reagent droplet comprises a barcoded bead. Methods of hydrogel formation are also described comprising selectively adding one or more reagents to one or more target cells, wherein the one or more reagents comprise a hydrogel precursor. In some embodiments, the methods comprise: flowing an emulsion comprising a plurality of droplets through a microfluidic device, wherein at least one droplet of the plurality of droplets comprises a target cell; flowing a plurality of reagent droplets comprising the one or more reagents through the microfluidic device; detecting via a detector a property of one or more droplets of the plurality of droplets; selectively merging one or more droplets of the plurality of droplets with one or more droplets of the plurality of reagent droplets based on the detection of the property to form the hydrogel within said one or more droplets of the plurality of droplets.

Also described herein are methods of hydrogel dissolution by selectively adding one or more reagents to one or more target cells in a reversible hydrogel, wherein the one or more reagents comprise a hydrogel reversing agent. In certain embodiments, the methods include: flowing an emulsion comprising a plurality of droplets through a microfluidic device, wherein at least one droplet of the plurality of droplets comprises a target cell in a reversible hydrogel; flowing a plurality of reagent droplets comprising the one or more reagents through the microfluidic device; detecting via a detector a property of one or more droplets of the plurality of droplets; and selectively merging one or more droplets of the plurality of droplets with one or more droplets of the plurality of reagent droplets based on the detection of the property to dissolve the hydrogel within said one or more droplets of the plurality of droplets.

The present disclosure also provides methods of selectively combining two or more populations of cells. In some embodiments, the methods comprise: flowing an emulsion comprising a first plurality of droplets comprising a first population of cells comprising at least one subpopulation of target cells through a microfluidic device, wherein each cell of the at least one subpopulation of target cells is optionally labeled with a first fluorescent moiety; flowing an emulsion comprising a second plurality of droplets comprising a second population of cells through the microfluidic device; detecting via a detector a property of one or more droplets of the first plurality of droplets; and selectively merging one or more droplets of the first plurality of droplets with one or more droplets of the second plurality of droplets based on the detection of the property. In some cases, the selective merging is performed by applying an electric field. In some cases, the fluorescent label is included and the optical property is a property of the fluorescent label. In other cases the optical property is the optical absorbance of the droplet. In other cases the optical property is the size of the droplet. In still other cases, the detecting is electrical detecting based on the conductivity of the droplet.

Further described herein are methods of selectively combining one or more populations of cells with one or more populations of microbes, viruses, or nucleic acids. In certain embodiments, the methods include: flowing an emulsion comprising a first plurality of droplets comprising a first population of cells comprising at least one subpopulation of target cells through a microfluidic device; flowing an emulsion comprising a second plurality of droplets comprising one or more populations of microbes, viruses, or nucleic acids through the microfluidic device; detecting via a detector a property of one or more droplets of the first plurality of droplets; and selectively merging one or more droplets of the first plurality of droplets with one or more droplets of the second plurality of droplets based on the detection of the property.

Also described herein are methods of selectively combining one or more populations of cells with one or more small molecules. In some embodiments, the methods comprise: flowing an emulsion comprising a first plurality of droplets comprising a first population of cells comprising at least one subpopulation of target cells through a microfluidic device; flowing an emulsion comprising a second plurality of droplets comprising one or more small molecules through the microfluidic device; detecting via a detector a property of one or more droplets of the first plurality of droplets; and selectively merging one or more droplets of the first plurality of droplets with one or more droplets of the second plurality of droplets based on the detection of the property.

Further provided herein are microfluidic devices comprising: a detector for detecting a property of one or more droplets of a plurality of droplets; an electrode; and an automated system, wherein the automated system applies an electric field via the electrode to selectively merge the one or more droplets of a first plurality of droplets with one or more droplets of a second plurality of droplets based on the detection of the property. In some cases the detector is an optical detector and the property is an optical property.

The present disclosure also describes systems comprising: the microfluidic devices described herein; a power source; and a controller, wherein the controller is configured to selectively enable or disable an electrical connection between the power source and the electrode thereby providing an active or inactive electrode respectively.

Also provided are kits comprising one or more of the microfluidic devices and systems disclosed herein. In some embodiments, the kits further comprise instructions to carry out the methods described herein.

### BRIEF DESCRIPTION OF THE DRAWINGS

The invention may be best understood from the following detailed description when read in conjunction with the accompanying drawings. Included in the drawings are the following figures:

FIG. 1 depicts a schematic of the experimental system used for selective merger. Re-injected droplets enter the microfluidic device downstream of reagent droplets, and pair in the long channel before the merger junction. Target drops (shown in gray) are merged with reagent droplets (shown in dark gray) based on their fluorescence, while negative drops (shown in light gray) remain unmerged. Lasers are focused with an objective lens onto flowing droplets to excite fluorescent molecules. Fluorescence is collected through the same objective and measured using photomultiplier tubes. Integrating under time trace data results in single fluorescence values for each droplet, which are plotted and used to assign gates. Droplets that fall within the desired gate are merged with reagents by applying a high voltage pulse to the salt-water electrode embedded on the microfluidic device. Drops that fall outside of the gates remain unmerged and are collected in the same tube.

FIG. 2, Panels A-F, illustrates embodiments efficient reagent addition using droplet coalescence. Panel A shows that the microfluidic device used for selective merger consists of an upstream reagent drop making and downstream drop re-injector. Larger reagent drops plug the channel, allowing smaller re-injected drops to catchup and efficiently pair in the leading channel. Selective merger is triggered with a salt-water electrode, merging the small and large droplets. Additional oil can be added to space droplets after the point of merger to ensure no off-target coalescence occurs. Panel B demonstrates that when the electrode remains off and no droplets are merged, it results in three populations—large CY5+ reagent droplets and small reinjected droplets containing two distinct fluorophore concentrations (FAM- and FAM+). Panel C shows that when the electrode is triggered on the high fluorescent population, merging it with reagents, results in four main populations—unmerged reagent drops (CY5+), unmerged low fluorescent drops (FAM−), and merged high fluorescent drops (CY5+ FAM+). Panel D shows the size profiles from droplet populations with and without selective merger. As expected, the emergence of a third, larger population of merged drops is observed. Panel E provides a quantification of the efficiency of selective merger using droplet cytometry to measure the fluorescence of each droplet. When the electrode is off, FAM droplets are not merged with CY5+ reagent droplets. When the electrode is triggered in response to high FAM signal, the merger with CY5+ reagent droplets occurs. The proportion of false positives (0.084%) and false negatives (1.58%) is calculated. The percentage of FAM- and FAM+ drops with respect to the total number of small drops is shown in parentheses.

FIG. 3, Panels A-C, illustrates targeting of subpopulations for single cell RNA-seq. Panel A shows that the stained cells are detected with a custom droplet cytometer, which is used to gate the desired population. Panel B shows that cells are merged with a stream of barcoded beads for mRNA capture and reverse transcription. Only merged cells are co-encapsulated with beads and reagents for cDNA synthesis. The stream immediately forms droplets at a T-junction downstream of the merging event. Panel C demonstrates that single cell RNA-seq and t-SNE clustering confirms that the desired subpopulations are targeted for sequencing. Merging all the cells with barcoded beads performs scRNA-seq on both B-cell and T-cells, while merging only T-cells targets that population.

FIG. 4, Panels A-D, illustrates targeting subpopulations for single cell DNA-seq. Panel A shows microscope image of the microfluidic device for selective merger of barcoded beads with cell lysate drops with annotated inlets and outlets. Panel B shows operation of the device showing bead reinjection, reagent drop formation, cell lysate drop reinjection, drop pairing and merger. Right: Higher magnification images showing bead-drop:cell-drop pairing and drop merger. Drops are colored dark gray (negative) and gray (positive) to aid in the figure interpretation. Panel C provides a bioinformatic analysis of single cell genome sequencing. Clustering of genomic mutations clearly identifies the cell of origin. Cells are classified accordingly. Panel D demonstrates that merging all drops with barcoded beads results in equal sequencing of both cancer cell types, while selectively merging with K562 cells results in targeted scDNA-seq of the correct population.

FIG. 5, Panels A and B provides an illustration of droplets being merged and physically sorted simultaneously.

FIG. 6 illustrates an embodiment in which droplets are merged and physically sorted simultaneously.

### DETAILED DESCRIPTION

Methods for selectively adding one or more reagents to one or more target molecules are provided. In certain aspects, the methods include: flowing an emulsion comprising a plurality of droplets through a microfluidic device, wherein at least one droplet of the plurality of droplets comprises a target molecule; flowing a plurality of reagent droplets comprising one or more reagents through the microfluidic device; detecting via a detector a property of one or more droplets of the plurality of droplets; and selectively merging one or more droplets of the plurality of droplets with one or more droplets of the plurality of reagent droplets based on the detection of the property. Systems, devices and kits for practicing the subject methods are also provided.

The subject methods and devices may find use in a wide variety of applications, such as increasing the accuracy and/or efficiency of single-cell sequencing, for example, by selectively adding one or more reagents to one or more target cells, selectively adding one or more hydrogel precursor reagents to one or more target cells to form hydrogels, selectively adding one or more hydrogel reversing agents to one or more target cells in a reversible hydrogel to dissolve hydrogels, selectively combining two or more populations of cells, selectively combining one or more populations of cells with one or more populations of microbes, viruses, nucleic acids, beads, beads with conjugated nucleic acids, and selectively combining one or more populations of cells with one or more small molecules. Assays which can be performed in accordance with the subject disclosure may be relevant for the detection of cancer or other diseases, monitoring disease progression, analyzing the DNA or RNA content of cells, and a variety of other applications in which it is desired to detect and/or quantify specific target cells.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets.

It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

As used in the claims, the term “comprising”, which is synonymous with “including”, “containing”, and “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, and/or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

With respect to the terms “comprising”, “consisting essentially of”, and “consisting of”, where one of these three terms is used herein, the presently disclosed subject matter can include the use of either of the other two terms.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

Analyzing every cell in a diverse sample provides insight into population-level heterogeneity. However, abundant cell types dominate the analysis and rare populations are scarcely represented in the data. To focus on specific cell types, the current paradigm is to physically isolate subsets of interest prior to analysis; however, this is difficult for rare populations as isolation often does not yield enough cells for analysis. The typical method to enrich is through physical separation of material by sorting. However, droplet sorting is difficult to implement reliably into commercial systems. Selective addition of reagents for targeted reactions has the ability to serve a similar functional role as sorting, but with greatly reduced engineering complexity. This enables the selection of drop subpopulations or recovery of specific material and can add enrichment functionality to commercial droplet workflows.

The instant disclosure demonstrates an alternative approach that selectively merges cells with reagents to achieve enzymatic reactions without having to first physically isolate cells using single-cell genome and transcriptome analysis of targeted cell subsets. Analyzing heterogeneous populations obviates the need for pre-enrichment and simplifies single cell workflows, making the method useful for other applications in single cell biology, combinatorial chemical synthesis, and drug screening.

Selective addition of fluids to droplets enables reactions to occur only in droplets of interest. Chemicals, small molecules, proteins/peptides, deoxyribonucleic acid (DNA), ribonucleic acid (RNA), oligonucleotides, buffers, enzymes, beads, cells, or virus can be selectively added to droplets of interest so that reactions (synthesis, polymerase chain reaction (PCR), multiple displacement amplification (MDA), reverse transcription, transposase mediated genomic insertions, transfection, transduction, transformation, etc.) occur only in those droplets.

Selectively adding one or more reagents: Provided herein are methods of selectively adding one or more reagents to one or more target molecules. In some embodiments, the methods include: flowing an emulsion comprising a plurality of droplets through a microfluidic device, wherein at least one droplet of the plurality of droplets comprises a target molecule; flowing a plurality of reagent droplets comprising one or more reagents through the microfluidic device; detecting via a detector a property of one or more droplets of the plurality of droplets; and selectively merging one or more droplets of the plurality of droplets with one or more droplets of the plurality of reagent droplets based on the detection of the property. In certain embodiments, the fluorescent moiety is a fluorescent molecule. In some embodiments, the fluorescent molecule is a dye molecule.

The terms “drop,” “droplet,” and “microdroplet” are used interchangeably herein, to refer to small, generally spherically structures, containing at least a first fluid phase, e.g., an aqueous phase (e.g., water). In some embodiments, the subject droplets entities have a dimension, e.g., a diameter, of or about 1.0 μm to 1000 μm, inclusive, such as 1.0 μm to 750 μm, 1.0 μm to 500 μm, 1.0 μm to 100 μm, 1.0 μm to 10 μm, or 1.0 μm to 5 μm, inclusive. In some embodiments, the droplets as described herein have a dimension, e.g., diameter, of or about 1.0 μm to 5 μm, 5 μm to 10 μm, 10 μm to 100 μm, 100 μm to 500 μm, 500 μm to 750 μm, or 750 μm to 1000 μm, inclusive. Furthermore, in some embodiments, the droplets as described herein have a volume ranging from about 1 fL to 100 nL, inclusive, such as from 1 fL to 100 pL, 1 fL to 10 pL, 1 fL to 1 pL, 1 fL to 100 fL, or 1 fL to 10 fL, inclusive. In some embodiments, dropelts as described herein have a volume of 1 fL to 10 fL, 10 fL to 100 fL, 100 fL to 1 pL, 1 pL to 10 pL, 10 pL to 100 pL or 100 pL to 1 nL, inclusive. For example, droplets according to the present disclosure generally range from 1 μm to 1000 μm, inclusive, in diameter. The droplets may be sphere shaped or they may have any other suitable shape, e.g., an ovular or oblong shape. In addition, the droplets as described herein may have a size and/or shape such that they may be produced in, on, or by a microfluidic device and/or flowed from or applied by a microfluidic device. Droplets according to the present disclosure may be used to encapsulate cells, nucleic acids (e.g., DNA), enzymes, reagents, and a variety of other components.

In some embodiments, the plurality of droplets comprise droplets of more than one type, e.g., more than one composition and/or size, such as a first type, e.g., a type containing one or more target molecules or cells of interest, and a second type, e.g., a type not containing one or more target molecules or cells of interest. In some embodiments, plurality of reagent droplets may contain one or more beads, such as magnetic beads and/or conductive beads.

In some embodiments, a surfactant may be used to stabilize the droplets. Accordingly, a droplet may involve a surfactant stabilized emulsion. Any convenient surfactant that allows for the desired reactions to be performed in the droplets may be used. In other embodiments, the droplet is not stabilized by surfactants or particles. The surfactant used depends on a number of factors such as the oil and aqueous phases (or other suitable immiscible phases, e.g., any suitable hydrophobic and hydrophilic phases) used for the emulsions. In selecting a surfactant, desirable properties that may be considered in choosing the surfactant may include one or more of the following: (1) the surfactant has low viscosity; (2) the surfactant is immiscible with the polymer used to construct the device, and thus it doesn't swell the device; (3) biocompatibility; (4) the assay reagents are not soluble in the surfactant; (5) the surfactant exhibits favorable gas solubility, in that it allows gases to come in and out; (6) the surfactant has a boiling point higher than the temperature used for PCR (e.g., 95° C.); (7) the emulsion stability; (8) that the surfactant stabilizes drops of the desired size; (9) that the surfactant has limited fluorescence properties; and (11) that the surfactant remains soluble over a range of temperatures.

Other surfactants can also be envisioned, including ionic surfactants. Other additives can also be included in the oil to stabilize the droplets, including polymers that increase droplet stability at temperatures above 35° C. The droplets described herein may be prepared as emulsions. The nature of the microfluidic channel (or a coating thereon), e.g., hydrophilic or hydrophobic, may be selected so as to be compatible with the type of emulsion being utilized at a particular point in a microfluidic workflow.

Emulsions may be generated using microfluidic devices as described in greater detail below. Microfluidic devices can form emulsions consisting of droplets that are extremely uniform in size. The droplet generation process may be accomplished by pumping two immiscible fluids, such as oil and water, into a junction. The junction shape, fluid properties (viscosity, interfacial tension, etc.), and flow rates influence the properties of the droplets generated but, for a relatively wide range of properties, droplets of controlled, uniform size can be generated using methods like T-junctions and flow focusing. To vary droplet size, the flow rates of the immiscible liquids may be varied since, for T-junction and flow focus methodologies over a certain range of properties, droplet size depends on total flow rate and the ratio of the two fluid flow rates. To generate an emulsion with microfluidic methods, the two fluids are normally loaded into two inlet reservoirs (syringes, pressure tubes) and then pressurized as needed to generate the desired flow rates (using syringe pumps, pressure regulators, gravity, etc.). This pumps the fluids through the device at the desired flow rates, thus generating droplet of the desired size and rate.

The emulsion may comprise the plurality of droplets in a suitable carrier fluid. As used herein, the term “carrier fluid” refers to a fluid configured or selected to contain one or more droplets in the emulsion as described herein. A carrier fluid may include one or more substances and may have one or more properties, e.g., viscosity, which allows it to be flowed through a microfluidic device or a portion thereof. In some embodiments, carrier fluids include, for example: oil or water, and may be in a liquid or gas phase.

In certain embodiments, the one or more reagents is a nucleic acid. In some embodiments, the nucleic acid is a ribonucleic acid (RNA) or a deoxyribonucleic acid (DNA). According to some embodiments, the DNA is an oligonucleotide or a plasmid DNA. For example, one population of droplets may contain competent E. coli cells and another population of droplets contains plasmid DNA with only 1% of the plasmid population carrying the desired insert, then selectively merging competent cell drops with a sub-population of target plasmid DNA enables the specific transformation of DNA with the target insert.

In some embodiments, the one or more reagents comprise a protein, a peptide, a buffer, an enzyme, a bead, an amplification mastermix, a PCR primer, an MDA reagent, a cell, a microbe and/or a chemical. According to some embodiments, the transposase enzyme is a Tn5 transposase enzyme. In certain embodiments, the bead is a barcoded bead. In certain embodiments, the microbe is a virus, a fungus or a bacterium. According to certain embodiments, the chemical is a small molecule, a hydrogel reversing agent or a hydrogel precursor. In some embodiments, the hydrogel precursor is tetramethylethylenediamine (TEMED), acrylamide, a calcium solution and a polyethylene glycol (PEG) diacrylate solution.

The terms “nucleic acid barcode sequence”, “nucleic acid barcode”, “barcode”, and the like as used herein refer to a nucleic acid having a sequence which can be used to identify and/or distinguish one or more first molecules to which the nucleic acid barcode is conjugated from one or more second molecules. Nucleic acid barcode sequences are typically short, e.g., about 5 to 20 bases in length, and may be conjugated to one or more target molecules of interest or amplification products thereof. Nucleic acid barcode sequences may be single or double stranded.

The terms “nucleic acid”, “nucleic acid molecule”, “oligonucleotide” and “polynucleotide” are used interchangeably and refer to a polymeric form of nucleotides of any length, either deoxyribonucleotides or ribonucleotides, or analogs thereof. The terms encompass, e.g., DNA, RNA and modified forms thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

The terms “polypeptide,” “peptide,” and “protein,” used interchangeably herein, refer to a polymeric form of amino acids of any length. NH₂ refers to the free amino group present at the amino terminus of a polypeptide. COOH refers to the free carboxyl group present at the carboxyl terminus of a polypeptide. In keeping with standard polypeptide nomenclature, J. Biol. Chem., 243 (1969), 3552-3559 is used.

Digital droplet multiple displacement amplification (ddMDA or MDA) generally refers to compartmentalizing the amplification reaction of nucleic acid template molecule(s) in a single droplet reaction compartment (e.g., microdroplet), which results in generally parallel or uniform amplification of the nucleic acid template molecules. In some embodiments, the amplification reaction refers to amplifying a single (or a very few, e.g., 10 or less, such as 5 or less) nucleic acid template molecule in a single microdroplet. In other embodiments, the amplification reaction may amplify multiple nucleic acid template molecules in a single nucleic acid template molecule. Since the nucleic acid template molecules are physically isolated from one another, the molecules are able to amplify to saturation without competing with other molecules for resources. This yields a generally uniform representation of all genomic sequences. In some embodiments, each single nucleic acid template molecule is physically isolated from other nucleic acid template molecules such that amplification of the nucleic acid template molecule occurs irrespective of what is occurring outside of the microdroplet. Furthermore, confining a single nucleic acid template molecule in a single microdroplet negates the need to share similar resources (e.g., primers, reagents, polymerase enzymes).

In some embodiments, the ddMDA reaction amplifies nucleic acid template molecules compartmentalized in reaction chambers (e.g., microdroplets) having picoliter interior volumes. In some examples, compartmentalizing reactions of the nucleic acid template molecules may be achieved by emulsifying the solution containing the nucleic acid template molecules to be amplified with oil with vigorous shaking. If a suitable surfactant is present, stable aqueous droplets suspended in oil are produced, each of which amplifies a single nucleic acid template molecule. Alternatively, in other examples, compartmentalizing reactions in microdroplets can be achieved by using microfluidic emulsification techniques.

As is described more fully herein, in various aspects the subject methods may be used to selectively merge a variety of target molecules such as components from cells or cells from biological samples. Components of interest include, but are not necessarily limited to, cells (e.g., circulating cells and/or circulating tumor cells), polynucleotides (e.g., DNA and/or RNA), polypeptides (e.g., peptides and/or proteins), and many other components that may be present in a biological sample. As used herein, the term “biological sample” encompasses a variety of sample types obtained from a variety of sources, which sample types contain biological material. For example, the term includes biological samples obtained from a mammalian subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, cells, serum, plasma, biological fluid, and tissue samples. “Biological sample” includes cells; biological fluids such as blood, cerebrospinal fluid, semen, saliva, and the like; bile; bone marrow; skin (e.g., skin biopsy); and antibodies obtained from an individual.

In certain embodiments, the one or more droplets of the plurality of droplets comprises a cell. In some embodiments, each droplet of the plurality of droplets comprises not more than one cell. For any of the methods described herein, according to certain embodiments, the target molecule is a rare cell. In some embodiments, the rare cell is a cancer cell. In some embodiments, the cancer cell is a circulating tumor cell. In some embodiments, the rare cell is a cell obtained from an in vitro fertilization procedure. In some embodiments, the rare cell is a cell obtained from an individual displaying genetic mosaicism. In some embodiments, the rare cell is a cell obtained from an organism produced using synthetic biology techniques. In some embodiments, the population of cells is a heterogeneous population of cells.

In some embodiments, the rare cell is present in a sample at a concentration of at least about 1 in 10² cells of the population of cells. In some embodiments, the rare cell is present in a sample at a concentration of at least about 1 in 10³ cells of the population of cells. In some embodiments, the rare cell is present in a sample at a concentration of at least about 1 in 10⁴ cells of the population of cells. In some embodiments, the rare cell is present in a sample at a concentration of at least about 1 in 10⁵ cells of the population of cells. In some embodiments, the rare cell is present in a sample at a concentration of at least about 1 in 10⁶ cells of the population of cells. In some embodiments, the rare cell is present in a sample at a concentration of at least about 1 in 10⁷ cells of the population of cells. In some embodiments, the rare cell is present in a sample at a concentration of at least about 1 in 10⁸ cells of the population of cells. In some embodiments, the rare cell is present in a sample at a concentration of at least about 1 in 10⁹ cells of the population of cells. In some embodiments, the rare cell is present in a sample at a concentration of at least about 1 in 10¹⁰ cells of the population of cells. In some embodiments, the rare cell is present in a sample at a concentration of at least about 1 in 10¹¹ cells of the population of cells. In some embodiments, the rare cell is present in a sample at a concentration of at least about 1 in 10¹² cells of the population of cells. In some embodiments, the rare cell is present in a sample at a concentration of at least about 1 in 10¹³ cells of the population of cells. In some embodiments, the rare cell is present in a sample at a concentration of at least about 1 in 10¹⁴ cells of the population of cells. In some embodiments, the rare cell is present in a sample at a concentration of at least about 1 in 10¹⁵ cells of the population of cells.

One or more lysing agents may also be added to the plurality of droplets, containing a cell, under conditions in which the cell(s) may be caused to burst, thereby releasing their genomes. The lysing agents may be added after the cells are encapsulated into the droplets. Any convenient lysing agent may be employed, such as proteinase K or cytotoxins. In particular embodiments, cells may be co-encapsulated in drops with lysis buffer containing detergents such as Triton X100 and/or proteinase K. The specific conditions in which the cell(s) may be caused to burst will vary depending on the specific lysing agent used. For example, if proteinase K is incorporated as a lysing agent, the discrete entities, e.g., droplets, may be heated to about 37-60° C. for about 20 min to lyse the cells and to allow the proteinase K to digest cellular proteins, after which they may be heated to about 95° C. for about 5-10 min to deactivate the proteinase K.

In certain aspects, cell lysis may also, or instead, rely on techniques that do not involve addition of lysing agent. For example, lysis may be achieved by mechanical techniques that may employ various geometric features to effect piercing, shearing, abrading, etc. of cells. Other types of mechanical breakage such as acoustic techniques may also be used. Further, thermal energy can also be used to lyse cells. Any convenient methods of effecting cell lysis may be employed in the methods described herein.

In some embodiments, it is desirable to concentrate reagents or other materials in the droplets. This can be accomplished using available techniques for concentrating reagents such as, for example, placing beads in the droplets that can bind certain components in the droplets, and then either removing the bead or the portion of the droplet that does not contain the bead to achieve a concentration increase.

Single-cell sequencing: Many commercial droplet microfluidic devices use barcoded beads to obtain single cell resolution. These devices cannot selectively pair beads with cells of interest and therefore must barcode and analyze every cell in a sample. Sequencing is therefore distributed over a large population instead of the cells of interest, reducing the information that can be obtained from important subpopulations. Commercial droplet workflows usually process tens of thousands of cells; an important population representing 1% of this total would mean 99% of the information generated is not informative. This is a significant loss in throughput and expense. Selective addition of beads to target cells allows sequencing power to be focused on the correct subpopulation.

Cells, DNA or RNA can be selectively combined with oligonucleotides for targeted PCR or RT-PCR. Cells can be selectively merged with oligo-dT to isolate polyadenylated mRNA from subpopulations. Target drops can be selective combined with Tn5 to perform targeted insertion of specific oligonucleotides into naked nucleic acids or cells.

An extension of this technology adds a simultaneous sorting step, which we also have reduced to practice. This is particularly important for some sequencing applications. For example, selective merger of Phi29 and reagents for multiple displacement amplification can be used to selectively amplify cells or molecules of interest. However, if the sorting step is not implemented, significant material from untargeted cells can end up in downstream processing. By adding simultaneous merge-sort, MDA reagents are added to selected droplets and separated, allowing ddMDA on subpopulations without contamination.

Described herein are methods of single-cell sequencing comprising selectively adding one or more reagents to one or more target cells. In certain embodiments, the methods comprise: flowing an emulsion comprising a plurality of droplets through a microfluidic device, wherein at least one droplet of the plurality of droplets comprises a single cell; flowing a plurality of reagent droplets comprising one or more reagents through the microfluidic device; detecting via a detector a property of one or more droplets of the plurality of droplets; selectively merging one or more droplets of the plurality of droplets with one or more droplets of the plurality of reagent droplets based on the detection of the property; and sequencing the selectively merged one or more droplets of the plurality of droplets. In certain embodiments, the method comprises single-cell RNA sequencing. According to some embodiments, the single cell labeled with a first fluorescent moiety is a rare cell. In some embodiments, the rare cell is a cancer cell.

Hydrogel formation via selectively adding one or more reagents: Chemicals can be selectively added to droplets of interest to form hydrogels. Formation of hydrogels via selective merger of precursors allows for easy removal of non-merged drops in downstream processing. Cells can be selectively combined with hydrogel precursors to capture the genomes of subpopulations. In this case, the target droplet is merged with material that allows for hydrogel formation within the droplet. For example ammonium persulfate can be selectively combined with tetramethylethylenediamine (TEMED) and acrylamide to form polyacrylamide hydrogels. Alginate can be selectively combined with calcium solutions to form alginate hydrogels. Droplets containing Polyethylene glycol-Thiol (PEG-4SH) solution can be selectively combined with PEG diacrylate solution to form PEG hydrogels.

Suitable hydrogel polymers may include, but are not limited to the following: actic acid, glycolic acid, acrylic acid, 1-hydroxyethyl methacrylate (HEMA), ethyl methacrylate (EMA), propylene glycol methacrylate (PEMA), acrylamide (AAM), N-vinylpyrrolidone, methyl methacrylate (MMA), glycidyl methacrylate (GDMA), glycol methacrylate (GMA), ethylene glycol, fumaric acid, and the like. Some hydrogel polymers require the use of a cross linking agent. Common cross linking agents include tetraethylene glycol dimethacrylate (TEGDMA) and N,N′-methylenebisacrylamide. The hydrogel droplets can be homopolymeric, or can comprise co-polymers of two or more of the aforementioned polymers. Exemplary hydrogel droplets include, but are not limited to, a copolymer of poly(ethylene oxide) (PEO) and poly(propylene oxide) (PPO); Pluronic™ F-127 (a difunctional block copolymer of PEO and PPO of the nominal formula EO₁₀₀-PO₆₅-EO₁₀₀, where EO is ethylene oxide and PO is propylene oxide); poloxamer 407 (a tri-block copolymer consisting of a central block of poly(propylene glycol) flanked by two hydrophilic blocks of poly(ethylene glycol)); a poly(ethylene oxide)-poly(propylene oxide)-poly(ethylene oxide) co-polymer with a nominal molecular weight of 12,500 Daltons and a PEO:PPO ratio of 2:1); a poly(N-isopropylacrylamide)-base hydrogel (a PNIPAAm-based hydrogel); a PNIPAAm-acrylic acid co-polymer (PNIPAAm-co-AAc); poly(2-hydroxyethyl methacrylate); poly(vinyl pyrrolidone); and the like.

The reverse methodology is also true: undesired drops can be targeted for destruction or elimination. The negative population can be selectively encapsulated in hydrogels, rendering it easy to discard or impervious to specific molecular reactions. The remaining population is significantly enriched in the material of interest. For example, droplets containing 1% agarose can be selectively combined with droplets containing no agarose so that the agarose no longer forms a hydrogel and contents trapped within the gel are released solely upon heating the drops.

Methods of hydrogel formation are also described comprising selectively adding one or more reagents to one or more target cells, wherein the one or more reagents comprise a hydrogel precursor. In some embodiments, the methods comprise: flowing an emulsion comprising a plurality of droplets through a microfluidic device, wherein at least one droplet of the plurality of droplets comprises a target cell; flowing a plurality of reagent droplets comprising the one or more reagents through the microfluidic device; detecting via a detector an property of one or more droplets of the plurality of droplets; and selectively merging one or more droplets of the plurality of droplets with one or more droplets of the plurality of reagent droplets based on the detection of the property to form the hydrogel within said one or more droplets of the plurality of droplets. In some embodiments, a polyacrylamide hydrogel is formed and at least one droplet of the plurality of droplets comprises a target cell labeled with a first fluorescent moiety and ammonium persulfate and the hydrogel precursors are tetramethylethylenediamine (TEMED) and acrylamide. According to some embodiments, an alginate hydrogel is formed and at least one droplet of the plurality of droplets comprises a target cell labeled with a first fluorescent moiety and alginate and the hydrogel precursor is a calcium solution.

In some embodiments, a PEG hydrogel is formed and at least one droplet of the plurality of droplets comprises a target cell labeled with a first fluorescent moiety and PEG-Thiol and the hydrogel precursor is a PEG diacrylate solution. In some embodiments, the target cell labeled with a first fluorescent moiety is a rare cell. In certain embodiments, the rare cell is a cancer cell.

The reverse methodology is also true: undesired drops can be targeted for destruction or elimination. The negative population can be selectively encapsulated in hydrogels, rendering it easy to discard or impervious to specific molecular reactions. The remaining population is significantly enriched in the material of interest. For example, droplets containing 1% agarose can be selectively combined with droplets containing no agarose so that the agarose no longer forms a hydrogel and contents trapped within the gel are released solely upon heating the drops. In some embodiments, the target cell labeled with a first fluorescent moiety is targeted for removal downstream.

Hydrogel dissolution via selectively adding one or more reagents: In addition, a reversible hydrogel (alginate, PEG hydrogel, polyacrylamide with a cleavable crosslinker) can be selectively combined with a reversing agent, such as ethylenediaminetetraacetic acid (EDTA), dithiothreitol (DTT) or ultraviolet (UV) light, to release contents. Also described herein are methods of hydrogel dissolution by selectively adding one or more reagents to one or more target cells in a reversible hydrogel, wherein the one or more reagents comprise a hydrogel reversing agent. In certain embodiments, the methods include: flowing an emulsion comprising a plurality of droplets through a microfluidic device, wherein at least one droplet of the plurality of droplets comprises a target cell labeled with a first fluorescent moiety in a reversible hydrogel; flowing a plurality of reagent droplets comprising the one or more reagents through the microfluidic device; detecting via an optical detector an optical property of one or more droplets of the plurality of droplets; and applying an electric field to selectively merge one or more droplets of the plurality of droplets with one or more droplets of the plurality of reagent droplets based on the detection of the first fluorescent moiety to dissolve the hydrogel within said one or more droplets of the plurality of droplets. In some embodiments, the reversible hydrogel is an alginate hydrogel with a cleavable crosslinker. In certain embodiments, the reversible hydrogel is a PEG hydrogel with a cleavable crosslinker. According to some embodiments, the reversible hydrogel is a polyacrylamide hydrogel with a cleavable crosslinker. In certain embodiments, the target cell labeled with a first fluorescent moiety is targeted for removal downstream. According to some embodiments, the reversible agent employed is EDTA. In some embodiments, the reversible agent is DTT. In certain embodiments, the reversible agent employed is UV light.

In some embodiments, for any of the methods disclosed herein at least one droplet of the plurality of droplets comprises a non-target molecule labeled with a second fluorescent moiety that is distinct from the first fluorescent moiety. In certain embodiments, at least one droplet of the plurality of droplets comprising the non-target molecule labeled with a second fluorescent moiety is not merged with one or more droplets of the plurality of reagent droplets based on the detection of the second fluorescent moiety. According to some embodiments, at least one droplet of the plurality of droplets comprises a non-target molecule that is not labeled with a fluorescent moiety. In some embodiments, at least one droplet of the plurality of droplets comprising the non-target molecule not labeled with a fluorescent moiety is not merged with one or more droplets of the plurality of reagent droplets. In some embodiments, the unmerged drops are removed downstream. In certain embodiments, the unmerged droplets are recovered. Such a recovery may be conducted by contacting one or more unmerged droplets with a portion of a device, such as a microfluidic orifice connected to a suction device for sucking one or more material, such as one or more solvent and/or reagent from one or more unmerged droplets. A microfluidic orifice may be inserted into an unmerged droplet and/or placed in proximity to an unmerged droplet, e.g., placed at a distance from an unmerged droplet having an order of magnitude of a droplet or smaller, for performing recovery from the droplet. According to certain embodiments, the recovered unmerged droplets are recycled such that the methods disclosed herein are repeated with the recovered droplets. In some embodiments, the recovered droplets are continuously recycled during performance of the instant methods.

According to some embodiments, applying the electric field selectively merges the one or more droplets of the plurality of droplets comprising the target molecule labeled with a first fluorescent moiety with one or more droplets of the plurality of reagent droplets based on the detection of the first fluorescent moiety. In some embodiments, the pluralities of merged and unmerged droplets are collected in one or more output containers. In certain embodiments, the pluralities of merged and unmerged droplets are collected in one or more output containers via one or more collection tubes comprising valves. In some embodiments, the methods further comprise incubating the collected plurality of droplets to allow reactions to occur in the one or more droplets of the plurality of droplets merged with the one or more droplets of the plurality of reagent droplets to produce one or more reaction products. According to some embodiments, the reaction comprises a chemical synthesis reaction, a PCR, an MDA, reverse transcription reaction, transfection reaction, transduction reaction and/or a transformation reaction. In certain embodiments, the methods disclosed herein further comprise rupturing the plurality of droplets and recovering the reaction products for analysis. In some embodiments, the reaction products are recovered via filtration. Such a recovery may also be conducted by contacting one or more merged droplets with a portion of a device, such as a microfluidic orifice connected to a suction device for sucking one or more material, such as one or more solvent and/or reagent from one or more merged droplets. A microfluidic orifice may be inserted into the merged droplet and/or placed in proximity to the merged droplet, e.g., placed at a distance from the merged droplet having an order of magnitude of a droplet or smaller, for performing recovery from the droplet. The portions or complete droplets recovered with any of the methods described herein can then be dispensed into a secondary container by flowing them from the array into the container. For example, using the suction method, individual droplets or droplet portions can be recovered from the droplet array and these portions flowed through a tube into a well on a well plate, where they are dispensed. This can be done one droplet at a time, dispensing each droplet into a separate well and thereby preserving the isolation of the droplets from one another. Once in the well, other operations can be perfumed on the droplet, such as propagating cells contained therein or performing biological reactions, such as enzyme-linked immunosorbent assay (ELISA), PCR, etc.

Embodiments of the methods may include modulating the environment of the plurality of droplets and thereby modulating the contents of the plurality of droplets, e.g., by adding and/or removing contents of the droplet. Such modulation may include modulating a temperature, pH, pressure, chemical composition, and/or radiation level of an environment of one or more droplets. Such modulation may also be of the immediate environment of one or more plurality of droplets, such as an emulsion in which the droplets are provided and/or one or more space, such as a conduit, channel, or container, within a microfluidic device. An immediate environment of a droplet which may be modulated may also include a fluid volume, such as a fluid flow, in which the droplet is provided. One or more droplets may also be stored in a modulated environment.

In some embodiments, the methods do not comprise physically sorting the plurality of droplets via a sorter. In other embodiments, the microfluidic device comprises a sorter, and wherein the method further comprises physically sorting the plurality of droplets via the sorter. In certain embodiments, the sorting of the plurality of droplets is performed simultaneously with selectively merging the one or more droplets of the plurality of droplets based on the detection of the first fluorescent moiety. In some embodiments, the sorting comprises physical separation of the plurality of droplets. In some embodiments, the one or more reagents is a multiple displacement amplification (MDA) reagent.

According to certain embodiments, the optical detector comprises an optical fiber configured to apply excitation energy to one or more droplets of the plurality of droplets. In some embodiments, the optical fiber is configured to collect a signal produced by the application of the excitation energy to one or more droplets of the plurality of droplets. In certain embodiments, the plurality of reagent droplets are formed upstream of the plurality of droplets in the microfluidic device. In some embodiments, the plurality of reagent droplets are formed in a T-junction upstream of the plurality of droplets. According to some embodiments, the optical detector comprises a detection region. In some embodiments, the microfluidic device comprises a merger junction, wherein the one or more droplets of the plurality of droplets is selectively merged with the one or more droplets of the plurality of reagent droplets by applying an electric field based on the detection of first fluorescent moiety. In some embodiments, the detection region is upstream of the merger junction. According to some embodiments, the one or more droplets of the plurality of droplets pair with the one or more droplets of the plurality of reagent droplets in a region in the microfluidic device that is upstream of the merger junction.

Selectively combining two or more populations of cells: Selective addition of cells to droplets enables cell-cell interaction to occur only in droplets of interest. For example, if one population of droplets contains cell (A) and another population of droplets contains a mixed population of cells (B), selective merging cell A drops with a specific sub-population of B drops enables the study of interactions between cell A and that specific subpopulation. The present disclosure provides methods of selectively combining two or more populations of cells. In some embodiments, the methods comprise: flowing an emulsion comprising a first plurality of droplets comprising a first population of cells comprising at least one subpopulation of target cells through a microfluidic device, wherein each cell of the at least one subpopulation of target cells; flowing an emulsion comprising a second plurality of droplets comprising a second population of cells through the microfluidic device; detecting via a detector a property of one or more droplets of the first plurality of droplets; and selectively merging one or more droplets of the first plurality of droplets with one or more droplets of the second plurality of droplets based on the detection of the property. In certain embodiments, the populations of cells are populations of microbial cells and the at least one subpopulation of target cells comprises a microbial cell that produces an antibiotic. In a non-limiting example, one population of droplets contains microbial cell mixture A and another population of droplets contains microbial cell mixture B with only 1% of the population producing antibiotics, therefore, selectively merging cell mixture A drops with the sub-population of drops containing only antibiotic-producing cell mixture B enables the study of identification of antibiotic-resistant cells from cell mixture A.

Selectively combining one or more populations of cells with one or more populations of microbes: Selective addition of microbes such as viruses to droplets enables microbe-cell interaction to occur only in droplets of interest. For example, to study if a cell's previous viral infection affects infection by a different virus, if one population of droplets contains virus A and another population of droplets contains cells in which 1% of the population was previously infected by virus B, selectively merging virus A drops with the sub-population of drops containing only cell with virus B infection enables the study of interactions between virus A-virus B. Alternatively, if one population of droplets contains microbial cell mixture A and another population of droplets contains mammalian cell mixture B and only 1% of the population is cancer cell, selectively merging cell mixture A drops with the sub-population of drops containing only cancer cells from mixture B enables the study of identification of microbial cells with anti-cancer activity. Described herein are methods of selectively combining one or more populations of cells with one or more populations of microbes. In certain embodiments, the methods include: flowing an emulsion comprising a first plurality of droplets comprising a first population of cells comprising at least one subpopulation of target cells through a microfluidic device; flowing an emulsion comprising a second plurality of droplets comprising one or more populations of microbes through the microfluidic device; detecting via a detector a property of one or more droplets of the first plurality of droplets; and selectively merging one or more droplets of the first plurality of droplets with one or more droplets of the second plurality of droplets based on the detection of the first fluorescent moiety. In some embodiments, the at least one subpopulation of target cells is labeled with a first fluorescent moiety was previously infected with a microbe that is distinct from the one or more populations of microbes in the second plurality of droplets. In certain embodiments, the microbe is a virus, a fungus or a bacterium.

Selective addition of naked DNA or RNA to droplets enables transformation or transfection reactions to occur only in droplets containing cells of interest. For example, if one population of droplets contains virus carrying a clustered regularly interspaced short palindromic repeats (CRISPR) system and another population of droplets contains cells, selectively merging virus drops with a sub-population of cell drops enables the CRISPR genome editing of specific cell types. Accordingly, in certain embodiments of the methods disclosed herein, the virus is a viral vector. In some embodiments, the viral vector further comprises a CRISPR system. In some embodiments, the viral vector further comprises a zinc finger nuclease (ZFN) system. In certain embodiments, the viral vector further comprises a transcription activator-like effector nuclease (TALEN) system.

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. “Gene editing” refers to a type of genetic engineering in which the nucleotide sequence of a target polynucleotide is changed through introduction of deletions, insertions, single stranded or double stranded breaks, or base substitutions to the polynucleotide sequence. In some embodiments, CRISPR-mediated gene editing utilizes the pathways of non-homologous end-joining (NHEJ) or homologous recombination to perform the edits. Gene regulation refers to increasing or decreasing the production of specific gene products such as protein or RNA. guide RNA sequences are used to target specific polynucleotide sequences for gene editing employing the CRISPR technique. Techniques of designing gRNAs and donor therapeutic polynucleotides for target specificity are well known in the art. For example, Doench, J., et al. Nature biotechnology 2014; 32(12): 1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans activating CRIPSPR RNA (tracrRNA); or a polynucleotide comprising CRISPR RNA (crRNA) and trans-activating CRIPSPR RNA (tracrRNA). In some embodiments, the gRNA is synthetic (Kelley, M. et al. (2016) J of Biotechnology 233 (2016) 74-83). CRISPR-Cas systems have been classified into six types (I through VI) and grouped into two-broad classes: the class 1 systems (types I, III, and IV) use a multi-protein complex to achieve interference, and the class 2 systems (types II, V, and VI) use a single-nuclease effector such as Cas9, Cas12, and Cas13 for interference.

As used herein, “TALEN” (transcription activator-like effector nucleases) refers to engineered nucleases that comprise a non-specific DNA-cleaving nuclease fused to a TALE DNA-binding domain, which can target DNA sequences and be used for genome editing. Boch (2011) Nature Biotech. 29: 135-6; and Boch et al. (2009) Science 326: 1509-12; Moscou et al. (2009) Science 326: 3501. TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence. To produce a TALEN, a TALE protein is fused to a nuclease (N), which is a wild-type or mutated Fok1 endonuclease.

Several mutations to Fok1 have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-1034; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Bio. 200: 96. The Fok1 domain functions as a dimer, requiring two constructs with unique DNA binding domains for sites in the target genome with proper orientation and spacing. Both the number of amino acid residues between the TALE DNA binding domain and the Fok1 cleavage domain and the number of bases between the two individual TALEN binding sites appear to be important parameters for achieving high levels of activity. Miller et al. (2011) Nature Biotech. 29: 143-8. TALENs specific to sequences in immune cells can be constructed using any method known in the art, including various schemes using modular components. Zhang et al. (2011) Nature Biotech. 29: 149-53; Geibler et al. (2011) PLoS ONE 6: e19509.

As used herein, “ZFN” (Zinc Finger Nuclease) refers to engineered nucleases that comprise a non-specific DNA-cleaving nuclease fused to a zinc finger DNA binding domain, which can target DNA sequences and be used for genome editing. Like a TALEN, a ZFN comprises a Fold nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160. A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5. ZFNs specific to sequences in immune cells can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; Guo et al. (2010) J. Mol. Bioi. 400: 96; U.S. Patent Publication 201110158957; and U.S. Patent Publication 2012/0060230.

Selectively combining one or more populations of cells with one or more small molecules: Selective addition of chemicals or drugs to droplets only containing cells of interest allows for testing cell responses to drugs within a subpopulation. For example, if one population of droplets contains a drug library and another population of droplets contains cells, then selectively merging drug drops with the sub-population of cell drops enables the identification of drugs with activity in specific cell subpopulations. Described herein are methods of selectively combining one or more populations of cells with one or more small molecules. In some embodiments, the methods comprise: flowing an emulsion comprising a first plurality of droplets comprising a first population of cells comprising at least one subpopulation of target cells through a microfluidic device; flowing an emulsion comprising a second plurality of droplets comprising one or more small molecules through the microfluidic device; detecting via a detector a property of one or more droplets of the first plurality of droplets; and selectively merging one or more droplets of the first plurality of droplets with one or more droplets of the second plurality of droplets based on the detection of the property.

In some embodiments, for any of the methods disclosed herein the subpopulation of target cells labeled with a first fluorescent moiety is a rare cell subpopulation. In some embodiments, the rare cell subpopulation is a cancer cell subpopulation. In certain embodiments, at least one droplet of the first plurality of droplets comprises a non-target molecule labeled with a second fluorescent moiety that is distinct from the first fluorescent moiety. According to some embodiments, the at least one droplet of the first plurality of droplets comprising the non-target molecule labeled with a second fluorescent moiety is not merged based on the detection of the second fluorescent moiety. In some embodiments, at least one droplet of the first plurality of droplets comprises a non-target molecule that is not labeled with a fluorescent moiety.

According to some embodiments, the at least one droplet of the first plurality of droplets comprising the non-target molecule not labeled with a fluorescent moiety is not merged. In some embodiments, the unmerged drops are removed downstream. In certain embodiments, the unmerged droplets are recovered. Such a recovery may be conducted by contacting one or more unmerged droplets with a portion of a device, such as a microfluidic orifice connected to a suction device for sucking one or more material, such as one or more solvent and/or reagent from one or more unmerged droplets. A microfluidic orifice may be inserted into an unmerged droplet and/or placed in proximity to an unmerged droplet, e.g., placed at a distance from an unmerged droplet having an order of magnitude of a droplet or smaller, for performing recovery from the droplet. According to certain embodiments, the recovered unmerged droplets are recycled such that the methods disclosed herein are repeated with the recovered droplets. In some embodiments, the recovered droplets are continuously recycled during performance of the instant methods.

In certain embodiments, applying the electric field selectively merges one or more droplets of the first plurality of droplets with one or more droplets of the second plurality of droplets based on the detection of the first fluorescent moiety. According to some embodiments, the plurality of droplets is collected in one or more output containers. In certain embodiments, the plurality of droplets is collected in one or more output containers via one or more collection tubes comprising valves.

In some embodiments, the methods further comprise incubating the collected plurality of droplets to allow reactions to occur in the one or more of the merged plurality of droplets to produce one or more reaction products. According to some embodiments, the reaction comprises a chemical synthesis reaction, a PCR, an MDA, reverse transcription reaction, transfection reaction, transduction reaction and/or a transformation reaction. In certain embodiments, the methods disclosed herein further comprise rupturing the plurality of droplets and recovering the reaction products for analysis. Such a recovery may also be conducted by contacting one or more merged droplets with a portion of a device, such as a microfluidic orifice connected to a suction device for sucking one or more material, such as one or more solvent and/or reagent from one or more merged droplets. A microfluidic orifice may be inserted into the merged droplet and/or placed in proximity to the merged droplet, e.g., placed at a distance from the merged droplet having an order of magnitude of a droplet or smaller, for performing recovery from the droplet. The portions or complete droplets recovered with any of the methods described herein can then be dispensed into a secondary container by flowing them from the array into the container. For example, using the suction method, individual droplets or droplet portions can be recovered from the droplet array and these portions flowed through a tube into a well on a well plate, where they are dispensed. This can be done one droplet at a time, dispensing each droplet into a separate well and thereby preserving the isolation of the droplets from one another. Once in the well, other operations can be perfumed on the droplet, such as propagating cells contained therein or performing biological reactions, such as enzyme-linked immunosorbent assay (ELISA), PCR, etc.

Embodiments of the methods may include modulating the environment of the first plurality of droplets and thereby modulating the contents of the first plurality of droplets, e.g., by adding and/or removing contents of the droplet. Such modulation may include modulating a temperature, pH, pressure, chemical composition, and/or radiation level of an environment of one or more droplets. Such modulation may also be of the immediate environment of one or more of the first plurality of droplets, such as an emulsion in which the droplets are provided and/or one or more space, such as a conduit, channel, or container, within a microfluidic device. An immediate environment of a droplet which may be modulated may also include a fluid volume, such as a fluid flow, in which the droplet is provided. One or more droplets may also be stored in a modulated environment.

In some embodiments, the methods do not comprise physically sorting the first plurality of droplets via a sorter. In other embodiments, the microfluidic device comprises a sorter, and wherein the method comprises physically sorting the first plurality of droplets via the sorter. In certain embodiments, the sorting of the first plurality of droplets is performed simultaneously with selectively merging the one or more droplets of the first plurality of droplets with one or more droplets of the second plurality of droplets based on the detection of the first fluorescent moiety. In some embodiments, the sorting comprises physical separation of the plurality of droplets.

In some embodiments, the optical detector comprises an optical fiber configured to apply excitation energy to one or more droplets of the first plurality of droplets. According to certain embodiments, the optical fiber is configured to collect a signal produced by the application of the excitation energy to one or more droplets of the first plurality of droplets. In some embodiments, the second plurality of droplets is formed upstream of the first plurality of droplets in the microfluidic device. In some embodiments, the second plurality of droplets is formed in a T-junction upstream of the first plurality of droplets. According to some embodiments, the optical detector comprises a detection region. In some embodiments, the microfluidic device comprises a merger junction, wherein the one or more droplets of the first plurality of droplets is selectively merged with the one or more droplets of the second plurality of droplets by applying an electric field based on the detection of the first fluorescent moiety. In certain embodiments, the detection region is upstream of the merger junction. According to some embodiments, the one or more droplets of the first plurality of droplets pair with the one or more of the second plurality of reagent droplets in a region in the microfluidic device that is upstream of the merger junction.

### Devices

Further provided herein are microfluidic devices comprising: an optical detector for detecting an optical property of one or more droplets of a plurality of droplets; an electrode; and an automated system, wherein the automated system applies an electric field via the electrode to selectively merge the one or more droplets of a first plurality of droplets with one or more droplets of a second plurality of droplets based on the detection of an optical property.

Accordingly, in some embodiments the methods disclosed herein include selectively merge the one or more droplets of a first plurality of droplets with one or more droplets of a second plurality of droplets based on the detection of a property using an automated system. In certain embodiments the methods disclosed herein include selectively merge the one or more droplets of a plurality of droplets with one or more reagent droplets based on the detection of an optical property using an automated system. Automated systems as disclosed may include one or more control units, e.g., control units including a central processing unit, to control one or more aspects of selectively merging droplets based on the detection of an optical property.

To analyze the fluorescence of a droplet, it is necessary to provide excitation light, e.g., in the form of a laser, and read the generated optical fiber configured to collect a signal produced by the application of excitation energy. In some embodiments of the invention, this can be accomplished using a single optical fiber that serves both to funnel the excitation light into the device and also collects the emitted light in the reverse direction. A drawback of this approach, however, is that the optical properties that are ideal for excitation light guidance may not be the same as for optical fiber configured to collect a signal produced by the application of excitation energy capture. For example, to excite a narrow beam, a fiber with a narrow tip is preferred, but to collect the largest number of emitted photons, a wide fiber with a large collecting cone angle is preferred. In these instances, multiple fibers can be used. For example, a narrow fiber can be used to provide a concentrated, excitation signal, while a wide fiber can collect the emitted fluorescent light.

According to some embodiments, the devices further comprise one or more droplet makers and one or more flow channels, wherein the one or more flow channels are fluidically connected to the one or more droplet makers and configured to receive one or more droplets therefrom. A variety of suitable droplet makers are known in the art, which may be used, e.g., droplet makers described in PCT Publication No. WO 2014/028378, the disclosure of which is incorporated by reference herein in its entirety and for all purposes. In some embodiments of the disclosed methods, microfluidic devices are utilized which include one or more droplet makers configured to form droplets from a fluid stream. Suitable droplet makers include selectively activatable droplet makers and the methods may include forming one or more droplets via selective activation of the droplet maker. The methods may also include forming droplets using a droplet maker, wherein the droplets include one or more entities which differ in composition.

In certain embodiments, the second plurality of droplets is formed upstream of the one or more droplets of the first plurality of droplets in the one or more flow channels. In some embodiments, the second plurality of droplets is formed in a T-junction upstream of the one or more droplets of the first plurality of droplets in the one or more flow channels.

According to some embodiments, the optical detector comprises a detection region. In some embodiments, the one or more flow channels comprise a merger junction, wherein the one or more droplets of the first plurality of droplets is selectively merged with the one or more droplets of the second plurality of droplets by activating the electrode based on the detection of an optical property. In some embodiments, the detection region is upstream of the merger junction.

According to some embodiments, the devices further comprise one or more output containers wherein the plurality of droplets is collected. In certain embodiments, the one or more output containers are fluidically connected to one or more collection tubes comprising valves. In some embodiments, the microfluidic devices do not comprise a sorter to physically sort the first plurality of droplets. In other embodiments, the microfluidic devices further comprise a sorter to physically sort the first plurality of droplets. In some embodiments, the sorter physically separates the first plurality of droplets, e.g., droplets having different types, e.g., different compositions and/or sizes, such as a first type, e.g., a type containing one or more cells of interest, and a second type, e.g., a type not containing one or more cells of interest.

As indicated above, embodiments of the disclosed subject matter employ systems and/or devices including microfluidic devices. Devices of the subject disclosure include all those described above in association with the subject methods. Microfluidic devices of this disclosure may be characterized in various ways.

By “operably connected” and “operably coupled”, as used herein, is meant connected in a specific way (e.g., in a manner allowing fluid, e.g., water, to move and/or electric power to be transmitted) that allows a disclosed system or device and its various components to operate effectively in the manner described herein.

As noted above, the microfluidic device may include one or more flow channels, e.g., flow channels which the plurality of droplets may pass into, out of, and/or through. The flow channels may comprise air channels and channels for flowing liquid such as flow channels. In certain embodiments, flow channels are one or more “micro” channel. Such channels may have at least one cross-sectional dimension on the order of a millimeter or smaller (e.g., less than or equal to about 1 millimeter). For certain applications, this dimension may be adjusted; in some embodiments the at least one cross-sectional dimension is about 500 micrometers or less. In some embodiments, the cross-sectional dimension is about 100 micrometers or less, or about 10 micrometers or less, and sometimes about 1 micrometer or less. A cross-sectional dimension is one that is generally perpendicular to the direction of centerline flow, although it should be understood that when encountering flow through elbows or other features that tend to change flow direction, the cross-sectional dimension in play need not be strictly perpendicular to flow. It should also be understood that in some embodiments, a micro-channel may have two or more cross-sectional dimensions such as the height and width of a rectangular cross-section or the major and minor axes of an elliptical cross-section. Either of these dimensions may be compared against sizes presented here. Note that micro-channels employed in this disclosure may have two dimensions that are grossly disproportionate—e.g., a rectangular cross-section having a height of about 100-200 micrometers and a width on the order or a centimeter or more. Of course, certain devices may employ channels in which the two or more axes are very similar or even identical in size (e.g., channels having a square or circular cross-section).

Microfluidic devices, in some embodiments of this disclosure, are fabricated using microfabrication technology. Such technology may be employed to fabricate integrated circuits (ICs), microelectromechanical devices (MEMS), display devices, and the like. Among the types of microfabrication processes that can be employed to produce small dimension patterns in microfluidic device fabrication are photolithography (including X-ray lithography, e-beam lithography, etc.), self-aligned deposition and etching technologies, anisotropic deposition and etching processes, self-assembling mask formation (e.g., forming layers of hydrophobic-hydrophilic copolymers), etc.

In view of the above, it should be understood that some of the principles and design features described herein can be scaled to larger devices and systems including devices and systems employing channels reaching the millimeter or even centimeter scale channel cross-sections. Thus, when describing some devices and systems as “microfluidic,” it is intended that the description apply equally, in certain embodiments, to some larger scale devices.

When referring to a microfluidic “device” it is generally intended to represent a single entity in which one or more channels, reservoirs, stations, etc. share a continuous substrate, which may or may not be monolithic. Aspects of microfluidic devices include the presence of one or more fluid flow paths, e.g., channels, having dimensions as discussed herein. A microfluidics “system” may include one or more microfluidic devices and associated fluidic connections, electrical connections, control/logic features, etc.

In various embodiments, microfluidic devices of this disclosure provide a continuous flow of a fluid medium. Fluid flowing through a channel in a microfluidic device exhibits many unique properties. Typically, the dimensionless Reynolds number is extremely low, resulting in flow that always remains laminar. Further, in this regime, two fluids joining will not easily mix, and diffusion alone may drive the mixing of two compounds.

In addition, the subject devices, in some embodiments, include one or more temperature and/or pressure control module. More specifically, a temperature control module may be one or more thermal cycler.

Microfluidic Elements: Microfluidic systems and devices according to the subject disclosure can contain one or more flow channels, such as microchannels, valves, pumps, reactors, mixers and other/or components. Some of these components and their general structures and dimensions are discussed below.

Various types of valves can be applied for flow control in microfluidic devices of this disclosure. These include but are not limited to passive valves and check valves (membrane, flap, bivalvular, leakage, etc.). Flow rate through these valves are dependent on various physical features of the valve such as surface area, size of flow channel, valve material, etc. Valves also have associated operational and manufacturing advantages/disadvantages that may be taken into consideration during design of a microfluidic device.

Embodiments of the subject devices include one or more micropumps. Micropumps, as with other microfluidic components, are subjected to manufacturing constraints. Typical considerations in pump design include treatment of bubbles, clogs, and durability. Micropumps which may be included in the subject devices include, but are not limited to electric equivalent pumps, fixed-stroke microdisplacement, peristaltic micromembrane and/or pumps with integrated check valves.

Macrodevices rely on turbulent forces such as shaking and stirring to mix reagents. In comparison, such turbulent forces are not practically attainable in microdevices, such as those of the present disclosure, and instead mixing in microfluidic devices is generally accomplished through diffusion. Since mixing through diffusion can be slow and inefficient, microstructures, such as those employed with the disclosed subject matter, are often designed to enhance the mixing process. These structures manipulate fluids in a way that increases interfacial surface area between the fluid regions, thereby speeding up diffusion. In certain embodiments, microfluidic mixers are employed. Such mixers may be provided upstream from, and in some cases integrated with, a microfluidic separation device and/or a sorter, of this disclosure.

In some embodiments, the devices and systems of the present disclosure include micromixers. Micromixers may be classified into two general categories: active mixers and passive mixers. Active mixers work by exerting active control overflow regions (e.g. varying pressure gradients, electric charges, etc.). Passive mixers do not require inputted energy and use only “fluid dynamics” (e.g. pressure) to drive fluid flow at a constant rate. One example of a passive mixer involves stacking two flow streams on top of one another separated by a plate. The flow streams are contacted with each other once the separation plate is removed. The stacking of the two liquids increases contact area and decreases diffusion length, thereby enhancing the diffusion process. Mixing and reaction devices can be connected to heat transfer systems if heat management is needed. As with macro-heat exchangers, micro-heat exchanges can either have co-current, counter-current, or cross-flow flow schemes. Microfluidic devices may have channel widths and depths between about 10 μm and about 10 cm. One channel structure includes a long main separation channel, and three shorter “offshoot” side channels terminating in either a buffer, sample, or waste reservoir. The separation channel can be several centimeters long, and the three side channels usually are only a few millimeters in length. Of course, the actual length, cross-sectional area, shape, and branch design of a microfluidic device depends on the application as well other design considerations such as throughput (which depends on flow resistance), velocity profile, residence time, etc.

Microfluidic devices described herein may include one or more electric field generators to perform certain steps of the methods described herein, such as selective droplet fusion. In certain embodiments, the electric fields are generated using metal electrodes. In particular embodiments, electric fields are generated using liquid electrodes. In certain embodiments, liquid electrodes include liquid electrode channels filled with a conducting liquid (e.g. salt water or buffer) and situated at positions in the microfluidic device where an electric field is desired. In particular embodiments, the liquid electrodes are energized using a power supply or high voltage amplifier. In some embodiments, the liquid electrode channel includes an inlet port so that a conducting liquid can be added to the liquid electrode channel. Such conducting liquid may be added to the liquid electrode channel, for example, by connecting a tube filled with the liquid to the inlet port and applying pressure. In particular embodiments, the liquid electrode channel also includes an outlet port for releasing conducting liquid from the channel. Liquid electrodes may find use, for example, where a material to be injected via application of an electric field is not charged.

In certain embodiments, the width of one or more of the microchannels of the microfluidic device (e.g., input microchannel, pairing microchannel, selective merger microchannel and/or a flow channel upstream or downstream of one or more of these channels) is 100 microns or less, e.g., 90 microns or less, 80 microns or less, 70 microns or less, 60 microns or less, 50 microns or less, e.g., 45 microns or less, 40 microns or less, 39 microns or less, 38 microns or less, 37 microns or less, 36 microns or less, 35 microns or less, 34 microns or less, 33 microns or less, 32 microns or less, 31 microns or less, 30 microns or less, 29 microns or less, 28 microns or less, 27 microns or less, 26 microns or less, 25 microns or less, 20 microns or less, 15 microns or less, or 10 microns or less. In some embodiments, the width of one or more of the above microchannels is from about 10 microns to about 15 microns, from about 15 microns to about 20 microns, from about 20 microns to about 25 microns, from about 25 microns to about 30 microns, from about 30 microns to about 35 microns, from about 35 microns to about 40 microns, from about 40 microns to about 45 microns, or from about 45 microns to about 50 microns, from about 50 microns to about 60 microns, from about 60 microns to about 70 microns, from about 70 microns to about 80 microns, from about 80 microns to about 90 microns, or from about 90 microns to about 100 microns. Additional descriptions of various microchannel structures and features which may be utilized in connection with the disclosed methods and devices are provided in PCT Publication No. WO 2014/028378, the disclosure of which is incorporated by reference herein in its entirety and for all purposes.

Methods of Fabrication: According to the disclosed embodiments, microfabrication processes differ depending on the type of materials used in the substrate and/or the desired production volume. For small volume production or prototypes, fabrication techniques include LIGA, powder blasting, laser ablation, mechanical machining, electrical discharge machining, photoforming, etc. Technologies for mass production of microfluidic devices may use either lithographic or master-based replication processes. Lithographic processes for fabricating substrates from silicon/glass include both wet and dry etching techniques commonly used in fabrication of semiconductor devices. Injection molding and hot embossing typically are used for mass production of plastic substrates.

Glass, Silicon and Other “Hard” Materials (Lithography, Etching, Deposition): According to embodiments of the disclosed subject matter, a combination of lithography, etching and/or deposition techniques may be used to make microcanals and microcavities out of glass, silicon and other “hard” materials. Technologies based on the above techniques may be applied in fabrication of devices in the scale of 0.1-500 micrometers.

Microfabrication techniques based on semiconductor fabrication processes are generally carried out in a clean room. The quality of the clean room is classified by the number of particles <4 μm in size in a cubic inch. Typical clean room classes for MEMS microfabrication may be 1000 to 10000.

In certain embodiments, photolithography may be used in microfabrication. In photolithography, a photoresist that has been deposited on a substrate is exposed to a light source through an optical mask. Conventional photoresist methods allow structural heights of up to 10-40 μm. If higher structures are needed, thicker photoresists such as SU-8, or polyimide, which results in heights of up to 1 mm, can be used.

After transferring the pattern on the mask to the photoresist-covered substrate, the substrate is then etched using either a wet or dry process. In wet etching, the substrate—area not protected by the mask—is subjected to chemical attack in the liquid phase. The liquid reagent used in the etching process depends on whether the etching is isotropic or anisotropic. Isotropic etching generally uses an acid to form three-dimensional structures such as spherical cavities in glass or silicon. Anisotropic etching forms flat surfaces such as wells and canals using a highly basic solvent. Wet anisotropic etching on silicon creates an oblique channel profile.

Dry etching involves attacking the substrate by ions in either a gaseous or plasma phase. Dry etching techniques can be used to create rectangular channel cross-sections and arbitrary channel pathways. Various types of dry etching that may be employed including physical, chemical, physico-chemical (e.g., RIE), and physico-chemical with inhibitor. Physical etching uses ions accelerated through an electric field to bombard the substrate's surface to “etch” the structures. Chemical etching may employ an electric field to migrate chemical species to the substrate's surface. The chemical species then reacts with the substrate's surface to produce voids and a volatile species.

In certain embodiments, deposition is used in microfabrication. Deposition techniques can be used to create layers of metals, insulators, semiconductors, polymers, proteins and other organic substances. Most deposition techniques fall into one of two main categories: physical vapor deposition (PVD) and chemical vapor deposition (CVD). In one approach to PVD, a substrate target is contacted with a holding gas (which may be produced by evaporation for example). Certain species in the gas adsorb to the target's surface, forming a layer constituting the deposit. In another approach commonly used in the microelectronics fabrication industry, a target containing the material to be deposited is sputtered with using an argon ion beam or other appropriately energetic source. The sputtered material then deposits on the surface of the microfluidic device. In CVD, species in contact with the target react with the surface, forming components that are chemically bonded to the object. Other deposition techniques include: spin coating, plasma spraying, plasma polymerization, dip coating, casting and Langmuir-Blodgett film deposition. In plasma spraying, a fine powder containing particles of up to 100 μm in diameter is suspended in a carrier gas. The mixture containing the particles is accelerated through a plasma jet and heated. Molten particles splatter onto a substrate and freeze to form a dense coating. Plasma polymerization produces polymer films (e.g. PMMA) from plasma containing organic vapors.

Once the microchannels, microcavities and other features have been etched into the glass or silicon substrate, the etched features are usually sealed to ensure that the microfluidic device is “watertight.” When sealing, adhesion can be applied on all surfaces brought into contact with one another. The sealing process may involve fusion techniques such as those developed for bonding between glass-silicon, glass-glass, or silicon-silicon.

Anodic bonding can be used for bonding glass to silicon. A voltage is applied between the glass and silicon and the temperature of the system is elevated to induce the sealing of the surfaces. The electric field and elevated temperature induces the migration of sodium ions in the glass to the glass-silicon interface. The sodium ions in the glass-silicon interface are highly reactive with the silicon surface forming a solid chemical bond between the surfaces. The type of glass used may have a thermal expansion coefficient near that of silicon (e.g. Pyrex Corning 7740).

Fusion bonding can be used for glass-glass or silicon-silicon sealing. The substrates are first forced and aligned together by applying a high contact force. Once in contact, atomic attraction forces (primarily van der Waals forces) hold the substrates together so they can be placed into a furnace and annealed at high temperatures. Depending on the material, temperatures used ranges between about 600 and 1100° C.

### Systems

The present disclosure also describes systems comprising: the microfluidic devices described herein; a power source; and a controller, wherein the controller is configured to selectively enable or disable an electrical connection between the power source and the electrode thereby providing an active or inactive electrode respectively.

### Kits

Also provided are kits comprising one or more of the microfluidic devices and systems disclosed herein. In some embodiments, the kits further comprise instructions to carry out the methods described herein.

These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., CD, DVD, Bluray, computer readable memory device (e.g., a flash memory drive), etc., on which the information has been recorded. Yet another means that may be present is a website address which may be used via the Internet to access the information at a removed site. Any convenient means may be present in the kits.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

1. A method of selectively adding one or more reagents to one or more target molecules comprising:
flowing an emulsion comprising a plurality of droplets through a microfluidic device, wherein at least one droplet of the plurality of droplets comprises a target molecule;
flowing a plurality of reagent droplets comprising one or more reagents through the microfluidic device;
detecting a property of one or more droplets of the plurality of droplets; and
selectively merging one or more droplets of the plurality of droplets with one or more droplets of the plurality of reagent droplets based on the detection of the property.
   2. The method of clause 1, wherein the property is an optical property.
   3. The method of clause 2, wherein the single cell is labelled with a first fluorescent moiety and the optical property is fluorescence of the first fluorescent moiety.
   4. The method of clause 2, wherein the optical property is absorbance.
   5. The method of clause 1, wherein the property is size.
   6. The method of clause 1, wherein the property is conductivity.
   7. The method of any one of clauses 1-6, wherein the selective merging comprises applying an electric field to selectively merge the one or more droplets with one or more droplets of the plurality of reagent droplets.
   8. The method of any one of clauses 1-6, wherein the selective merging comprises stream merging of the one or more droplets with one or more droplets of the plurality of reagent droplets.
   9. The method of any one of clauses 1-6, wherein the selective merging comprises pico-injection.
   10. The method of any one of clauses 1-6, wherein the selective merging comprises triple-emulsion coalescence.
   11. The method of any one of clauses 1-9, wherein the one or more reagents is a nucleic acid.
   12. The method of clause 10, wherein the nucleic acid is a ribonucleic acid (RNA).
   13. The method of clause 10, wherein the nucleic acid is a deoxyribonucleic acid (DNA).
   14. The method of clause 12, wherein the DNA is an oligonucleotide.
   15. The method of clause 12, wherein the DNA is plasmid DNA.
   16. The method of any one of clauses 1-9, wherein the one or more reagents is a protein.
   17. The method of any one of clauses 1-9, wherein the one or more reagents is a peptide.
   18. The method of any one of clauses 1-9, wherein the one or more reagents is a buffer.
   19. The method of any one of clauses 1-9, wherein the one or more reagents is an enzyme.
   20. The method of clause 18, wherein the enzyme is a transposase enzyme.
   21. The method of any one of clauses 1-9, wherein the one or more reagents is a bead.
   22. The method of clause 20, wherein the bead is a barcoded bead.
   23. The method of any one of clauses 1-9, wherein the one or more reagents is an amplification mastermix.
   24. The method of any one of clauses 1-9, wherein the one or more reagents is a polymerase chain reaction (PCR) primer.
   25. The method of any one of clauses 1-9, wherein the one or more reagents is a multiple displacement amplification (MDA) reagent.
   26. The method of any one of clauses 1-9, wherein the one or more reagents is a cell.
   27. The method of any one of clauses 1-9, wherein the one or more reagents is a microbe.
   28. The method of clause 26, wherein the microbe is a virus.
   29. The method of clause 26, wherein the microbe is a fungus.
   30. The method of clause 26, wherein the microbe is a bacterium.
   31. The method of any one of clauses 1-9, wherein the one or more reagents is a chemical.
   32. The method of clause 30, wherein the chemical is a small molecule.
   33. The method of clause 30, wherein the chemical is a hydrogel reversing agent.
   34. The method of clause 30, wherein the chemical is a hydrogel precursor.
   35. The method of clause 31, wherein the hydrogel precursor is tetramethylethylenediamine (TEMED).
   36. The method of clause 31, wherein the hydrogel precursor is acrylamide.
   37. The method of clause 31, wherein the hydrogel precursor is a calcium solution.
   38. The method of clause 31, wherein the hydrogel precursor is a polyethylene glycol (PEG) diacrylate solution.
   39. The method of any one of clauses 1-37, wherein the one or more droplets of the plurality of droplets comprises a cell.
   40. The method of clause 38, wherein each droplet of the plurality of droplets comprises not more than one cell.
   41. The method of clause 38 or 39, wherein the target molecule is a rare cell.
   42. The method of clause 40, wherein the rare cell is a cancer cell.
   43. A method of single-cell sequencing comprising selectively adding one or more reagents to one or more target cells, the method comprising:
flowing an emulsion comprising a plurality of droplets through a microfluidic device, wherein at least one droplet of the plurality of droplets comprises a single cell;
flowing a plurality of reagent droplets comprising one or more reagents through the microfluidic device;
detecting a property of one or more droplets of the plurality of droplets;
applying an electric field to selectively merge one or more droplets of the plurality of droplets with one or more droplets of the plurality of reagent droplets based on the detection of the property; and
sequencing the selectively merged one or more droplets of the plurality of droplets.
   44. The method of clause 42, wherein the property is an optical property.
   45. The method of clause 43, wherein the single cell is labelled with a first fluorescent moiety and the optical property is fluorescence of the first fluorescent moiety.
   46. The method of clause 43, wherein the optical property is absorbance.
   47. The method of clause 42, wherein the property is size.
   48. The method of clause 42, wherein the property is conductivity.
   49. The method of any one of clauses 42-47, wherein the selective merging comprises applying an electric field to selectively merge the one or more droplets with one or more droplets of the plurality of reagent droplets.
   50. The method of any one of clauses 42-47, wherein the selective merging comprises stream merging of the one or more droplets with one or more droplets of the plurality of reagent droplets.
   51. The method of any one of clauses 42-47, wherein the selective merging comprises pico-injection.
   52. The method of any one of clauses 42-50, wherein the method comprises single-cell RNA sequencing.
   53. The method of any one of clauses 42-51, wherein the single cell labeled with a first fluorescent moiety is a rare cell.
   54. The method of clause 52, wherein the rare cell is a cancer cell.
   55. A method of hydrogel formation comprising selectively adding one or more reagents to one or more target cells, wherein the one or more reagents comprise a hydrogel precursor, the method comprising:
flowing an emulsion comprising a plurality of droplets through a microfluidic device, wherein at least one droplet of the plurality of droplets comprises a target cell;
flowing a plurality of reagent droplets comprising the one or more reagents through the microfluidic device;
detecting a property of one or more droplets of the plurality of droplets; and
selectively merging one or more droplets of the plurality of droplets with one or more droplets of the plurality of reagent droplets based on the detection of the property to form the hydrogel within said one or more droplets of the plurality of droplets.
   56. The method of clause 54, wherein the property is an optical property.
   57. The method of clause 55, wherein the single cell is labelled with a first fluorescent moiety and the optical property is fluorescence of the first fluorescent moiety.
   58. The method of clause 55, wherein the optical property is absorbance.
   59. The method of clause 54, wherein the property is size.
   60. The method of clause 54, wherein the property is conductivity.
   61. The method of any one of clauses 54-59, wherein the selective merging comprises applying an electric field to selectively merge the one or more droplets with one or more droplets of the plurality of reagent droplets.
   62. The method of any one of clauses 54-59, wherein the selective merging comprises stream merging of the one or more droplets with one or more droplets of the plurality of reagent droplets.
   63. The method of any one of clauses 54-59, wherein the selective merging comprises pico-injection.
   64. The method of any one of clauses 54-62, wherein a polyacrylamide hydrogel is formed and wherein at least one droplet of the plurality of droplets comprises a target cell labeled with a first fluorescent moiety and ammonium persulfate and wherein the hydrogel precursors are tetramethylethylenediamine (TEMED) and acrylamide.
   65. The method of any one of clauses 54-63, wherein an alginate hydrogel is formed and wherein at least one droplet of the plurality of droplets comprises a target cell labeled with a first fluorescent moiety and alginate and wherein the hydrogel precursor is a calcium solution.
   66. The method of any one of clauses 54-64, wherein a PEG hydrogel is formed and wherein at least one droplet of the plurality of droplets comprises a target cell labeled with a first fluorescent moiety and PEG-Thiol and wherein the hydrogel precursor is a PEG diacrylate solution.
   67. The method of any one of clauses 54-65, wherein the target cell labeled with a first fluorescent moiety is a rare cell.
   68. The method of clause 66, wherein the rare cell is a cancer cell.
   69. The method of any one of clauses 54-67, wherein the target cell labeled with a first fluorescent moiety is targeted for removal downstream.
   70. A method of hydrogel dissolution by selectively adding one or more reagents to one or more target cells in a reversible hydrogel, wherein the one or more reagents comprise a hydrogel reversing agent, the method comprising:
flowing an emulsion comprising a plurality of droplets through a microfluidic device, wherein at least one droplet of the plurality of droplets comprises a target cell in a reversible hydrogel;
flowing a plurality of reagent droplets comprising the one or more reagents through the microfluidic device;
detecting a property of one or more droplets of the plurality of droplets; and
selectively merging one or more droplets of the plurality of droplets with one or more droplets of the plurality of reagent droplets based on the detection of the property to dissolve the hydrogel within said one or more droplets of the plurality of droplets.
   71. The method of clause 69, wherein the property is an optical property.
   72. The method of clause 70, wherein the single cell is labelled with a first fluorescent moiety and the optical property is fluorescence of the first fluorescent moiety.
   73. The method of clause 70, wherein the optical property is absorbance.
   74. The method of clause 69, wherein the property is size.
   75. The method of clause 69, wherein the property is conductivity.
   76. The method of any one of clauses 69-74, wherein the selective merging comprises applying an electric field to selectively merge the one or more droplets with one or more droplets of the plurality of reagent droplets.
   77. The method of any one of clauses 69-74, wherein the selective merging comprises stream merging of the one or more droplets with one or more droplets of the plurality of reagent droplets.
   78. The method of any one of clauses 69-74, wherein the selective merging comprises pico-injection.
   79. The method of any one of clauses 69-77, wherein the reversible hydrogel is an alginate hydrogel with a cleavable crosslinker.
   80. The method of any one of clauses 69-78, wherein the reversible hydrogel is a PEG hydrogel with a cleavable crosslinker.
   81. The method of any one of clauses 69-79, wherein the reversible hydrogel is a polyacrylamide hydrogel with a cleavable crosslinker.
   82. The method of any one of clauses 69-80, wherein the hydrogel reversing agent is ethylenediaminetetraacetic acid (EDTA).
   83. The method of any one of clauses 69-80, wherein the hydrogel reversing agent is dithiothreitol (DTT).
   84. The method of any one of clauses 69-80, wherein the hydrogel reversing agent is ultraviolet (UV) light.
   85. The method of any one of clauses 69-83, wherein the target cell labeled with a first fluorescent moiety is targeted for removal downstream.
   86. The method of any one of clauses 1-84, wherein at least one droplet of the plurality of droplets comprises a non-target molecule labeled with a second fluorescent moiety that is distinct from the first fluorescent moiety.
   87. The method of clause 85, wherein the at least one droplet of the plurality of droplets comprising the non-target molecule labeled with a second fluorescent moiety is not merged with one or more droplets of the plurality of reagent droplets based on the detection of the second fluorescent moiety.
   88. The method of clause any one of clauses 1-84, wherein at least one droplet of the plurality of droplets comprises a non-target molecule that is not labeled with a fluorescent moiety.
   89. The method of clause 87, wherein the at least one droplet of the plurality of droplets comprising the non-target molecule not labeled with a fluorescent moiety is not merged with one or more droplets of the plurality of reagent droplets.
   90. The method of clause 86 or 88, wherein the unmerged droplets are removed downstream.
   91. The method of clause 86 or 88, wherein the unmerged droplets are recovered.
   92. The method of clause 90, wherein the recovered unmerged droplets are recycled such that the method of any one of clauses 1-88 is repeated with the recovered droplets.
   93. The method of clause 91, wherein the recovered droplets are continuously recycled during performance of the method.
   94. The method of any one of clauses 1-92, wherein applying the electric field selectively merges the one or more droplets of the plurality of droplets comprising the target molecule labeled with a first fluorescent moiety with one or more droplets of the plurality of reagent droplets based on the detection of the first fluorescent moiety.
   95. The method of any one of clauses 86, 88 or 93, wherein the plurality of droplets is collected in one or more output containers.
   96. The method of clause 94, wherein the plurality of droplets is collected in one or more output containers via one or more collection tubes comprising valves.
   97. The method of clause 94 or 95, further comprising incubating the collected plurality of droplets to allow reactions to occur in the one or more droplets of the plurality of droplets merged with the one or more droplets of the plurality of reagent droplets to produce one or more reaction products.
   98. The method of clause 96, wherein the reaction is a chemical synthesis reaction.
   99. The method of clause 96, wherein the reaction comprises a polymerase chain reaction (PCR).
   100. The method of clause 96, wherein the reaction comprises a multiple displacement amplification (MDA).
   101. The method of clause 96, wherein the reaction comprises a reverse transcription reaction.
   102. The method of clause 96, wherein the reaction comprises a transfection reaction.
   103. The method of clause 96, wherein the reaction comprises a transduction reaction.
   104. The method of clause 96, wherein the reaction comprises a transformation reaction.
   105. The method of any one of clauses 95-103, further comprising rupturing the plurality of droplets and recovering the reaction products for analysis.
   106. The method of clause 104, wherein the reaction products are recovered via filtration.
   107. The method of any one of clauses 1-105, wherein the method does not comprise physically sorting the plurality of droplets via a sorter.
   108. The method of any one of clauses 1-105, wherein the microfluidic device comprises a sorter, and wherein the method further comprises physically sorting the plurality of droplets via the sorter.
   109. The method of clause 107, wherein the sorting of the plurality of droplets is performed simultaneously with selectively merging the one or more droplets of the plurality of droplets based on the detection of the first fluorescent moiety.
   110. The method of clause 107 or 108, wherein the sorting comprises physical separation of the plurality of droplets.
   111. The method of any one of clauses 107-109, wherein the one or more reagents is a multiple displacement amplification (MDA) reagent.
   112. The method of any one of clauses 1-110, wherein the optical detector comprises an optical fiber configured to apply excitation energy to one or more droplets of the plurality of droplets.
   113. The method of clause 111, wherein the optical fiber is configured to collect a signal produced by the application of the excitation energy to one or more droplets of the plurality of droplets.
   114. The method of any one of clauses 1-112, wherein the plurality of reagent droplets are formed upstream of the plurality of droplets in the microfluidic device.
   115. The method of clause 113, wherein the plurality of reagent droplets are formed in a T-junction upstream of the plurality of droplets.
   116. The method of any one of clauses 1-114, wherein the optical detector comprises a detection region.
   117. The method of any one of clauses 1-115, wherein the microfluidic device comprises a merger junction, wherein the one or more droplets of the plurality of droplets is selectively merged with the one or more droplets of the plurality of reagent droplets by applying an electric field based on the detection of first fluorescent moiety.
   118. The method of clause 116, wherein the detection region is upstream of the merger junction.
   119. The method of clause 116 or 117, wherein the one or more droplets of the plurality of droplets pair with the one or more droplets of the plurality of reagent droplets in a region in the microfluidic device that is upstream of the merger junction.
   120. A method of selectively combining two or more populations of cells comprising:
flowing an emulsion comprising a first plurality of droplets comprising a first population of cells comprising at least one subpopulation of target cells through a microfluidic device, wherein each cell of the at least one subpopulation of target cells is labeled with a first fluorescent moiety;
flowing an emulsion comprising a second plurality of droplets comprising a second population of cells through the microfluidic device;
detecting a property of one or more droplets of the first plurality of droplets; and
selectively merging one or more droplets of the first plurality of droplets with one or more droplets of the second plurality of droplets based on the detection of the property.
   121. The method of clause 119, wherein the property is an optical property.
   122. The method of clause 120, wherein the single cell is labelled with a first fluorescent moiety and the optical property is fluorescence of the first fluorescent moiety.
   123. The method of clause 120, wherein the optical property is absorbance.
   124. The method of clause 119, wherein the property is size.
   125. The method of clause 119, wherein the property is conductivity.
   126. The method of any one of clauses 119-124, wherein the selective merging comprises applying an electric field to selectively merge the one or more droplets with one or more droplets of the plurality of reagent droplets.
   127. The method of any one of clauses 119-124, wherein the selective merging comprises stream merging of the one or more droplets with one or more droplets of the plurality of reagent droplets.
   128. The method of any one of clauses 119-124, wherein the selective merging comprises pico-injection.
   129. The method of any one of clauses 119-127, wherein the populations of cells are populations of microbial cells and wherein the at least one subpopulation of target cells comprises a microbial cell that produces an antibiotic.
   130. A method of selectively combining one or more populations of cells with one or more populations of microbes comprising:
flowing an emulsion comprising a first plurality of droplets comprising a first population of cells comprising at least one subpopulation of target cells through a microfluidic device, wherein each cell of the at least one subpopulation of target cells is labeled with a first fluorescent moiety;
flowing an emulsion comprising a second plurality of droplets comprising one or more populations of microbes through the microfluidic device;
detecting a property of one or more droplets of the first plurality of droplets; and
selectively merging one or more droplets of the first plurality of droplets with one or more droplets of the second plurality of droplets based on the detection of the property.
   131. The method of clause 129, wherein the property is an optical property.
   132. The method of clause 130, wherein the single cell is labelled with a first fluorescent moiety and the optical property is fluorescence of the first fluorescent moiety.
   133. The method of clause 130, wherein the optical property is absorbance.
   134. The method of clause 129, wherein the property is size.
   135. The method of clause 129, wherein the property is conductivity.
   136. The method of any one of clauses 129-134, wherein the selective merging comprises applying an electric field to selectively merge the one or more droplets with one or more droplets of the plurality of reagent droplets.
   137. The method of any one of clauses 129-134, wherein the selective merging comprises stream merging of the one or more droplets with one or more droplets of the plurality of reagent droplets.
   138. The method of any one of clauses 129-134, wherein the selective merging comprises pico-injection.
   139. The method of any one of clauses 129-137, wherein the at least one subpopulation of target cells is labeled with a first fluorescent moiety was previously infected with a microbe that is distinct from the one or more populations of microbes in the second plurality of droplets.
   140. The method of any one of clauses 129-138, wherein the microbe is a bacterium.
   141. The method of clause 139, wherein the microbe is a fungus.
   142. The method of clause 139, wherein the microbe is a virus.
   143. The method of clause 141, wherein the virus is a viral vector.
   144. The method of clause 142, wherein the viral vector further comprises a clustered regularly interspaced short palindromic repeats (CRISPR) system.
   145. The method of clause 142, wherein the viral vector further comprises a zinc finger nuclease (ZFN) system.
   146. The method of clause 142, wherein the viral vector further comprises a transcription activator-like effector nuclease (TALEN) system.
   147. A method of selectively combining one or more populations of cells with one or more small molecules comprising:
flowing an emulsion comprising a first plurality of droplets comprising a first population of cells comprising at least one subpopulation of target cells through a microfluidic device;
flowing an emulsion comprising a second plurality of droplets comprising one or more small molecules through the microfluidic device;
detecting a property of one or more droplets of the first plurality of droplets; and
selectively merging one or more droplets of the first plurality of droplets with one or more droplets of the second plurality of droplets based on the detection of the property.
   148. The method of clause 146, wherein the property is an optical property.
   149. The method of clause 147, wherein the single cell is labelled with a first fluorescent moiety and the optical property is fluorescence of the first fluorescent moiety.
   150. The method of clause 147, wherein the optical property is absorbance.
   151. The method of clause 146, wherein the property is size.
   152. The method of clause 146, wherein the property is conductivity.
   153. The method of any one of clauses 146-151, wherein the selective merging comprises applying an electric field to selectively merge the one or more droplets with one or more droplets of the plurality of reagent droplets.
   154. The method of any one of clauses 146-151, wherein the selective merging comprises stream merging of the one or more droplets with one or more droplets of the plurality of reagent droplets.
   155. The method any one of clauses 146-151, wherein the selective merging comprises pico-injection.
   156. The method of any one of clauses 146-154, wherein the subpopulation of target cells labeled with a first fluorescent moiety is a rare cell subpopulation.
   157. The method of any one of clauses 146-155, wherein the rare cell subpopulation is a cancer cell subpopulation.
   158. The method of any one of clauses 119-156, wherein at least one droplet of the first plurality of droplets comprises a non-target molecule labeled with a second fluorescent moiety that is distinct from the first fluorescent moiety.
   159. The method of clause 157, wherein the at least one droplet of the first plurality of droplets comprising the non-target molecule labeled with a second fluorescent moiety is not merged based on the detection of the second fluorescent moiety.
   160. The method of any one of clauses 119-156, wherein at least one droplet of the first plurality of droplets comprises a non-target molecule that is not labeled with a fluorescent moiety.
   161. The method of clause 159, wherein the at least one droplet of the first plurality of droplets comprising the non-target molecule not labeled with a fluorescent moiety is not merged.
   162. The method of clause 158 or 160, wherein the unmerged droplets are removed downstream.
   163. The method of clause 158 or 160, wherein the unmerged droplets are recovered.
   164. The method of clause 162, wherein the recovered unmerged droplets are recycled such that the method of any one of clauses 119-160 is repeated with the recovered droplets.
   165. The method of clause 163, wherein the recovered droplets are continuously recycled during performance of the method.
   166. The method of any one of clauses 146-164, wherein applying the electric field selectively merges one or more droplets of the first plurality of droplets with one or more droplets of the second plurality of droplets based on the detection of the first fluorescent moiety.
   167. The method of any one of clauses 146-165, wherein the plurality of droplets is collected in one or more output containers.
   168. The method of clause 166, wherein the plurality of droplets is collected in one or more output containers via one or more collection tubes comprising valves.
   169. The method of clause 166 or 167, further comprising incubating the collected plurality of droplets to allow reactions to occur in the one or more of the merged plurality of droplets to produce one or more reaction products.
   170. The method of clause 168, wherein the reaction comprises a chemical synthesis reaction.
   171. The method of clause 168, wherein the reaction comprises a polymerase chain reaction (PCR).
   172. The method of clause 168, wherein the reaction comprises a multiple displacement amplification (MDA).
   173. The method of clause 168, wherein the reaction comprises a reverse transcription reaction.
   174. The method of clause 168, wherein the reaction comprises a transfection reaction.
   175. The method of clause 168, wherein the reaction comprises a transduction reaction.
   176. The method of clause 168, wherein the reaction comprises a transformation reaction.
   177. The method of any one of clauses 167-175, further comprising rupturing the plurality of droplets and recovering the reaction products for analysis.
   178. The method of any one of clauses 119-176, wherein the method does not comprise physically sorting the first plurality of droplets via a sorter.
   179. The method of any one of clauses 119-176, wherein the microfluidic device comprises a sorter, and wherein the method comprises physically sorting the first plurality of droplets via the sorter.
   180. The method of clause 178, wherein the sorting of the first plurality of droplets is performed simultaneously with selectively merging the one or more droplets of the first plurality of droplets with one or more droplets of the second plurality of droplets based on the detection of the first fluorescent moiety.
   181. The method of clause 178 or 179, wherein the sorting comprises physical separation of the plurality of droplets.
   182. The method of any one of clauses 119-180, wherein the optical detector comprises an optical fiber configured to apply excitation energy to one or more droplets of the first plurality of droplets.
   183. The method of clause 181, wherein the optical fiber is configured to collect a signal produced by the application of the excitation energy to one or more droplets of the first plurality of droplets.
   184. The method of any one of clauses 119-182, wherein the second plurality of droplets is formed upstream of the first plurality of droplets in the microfluidic device.
   185. The method of clause 183, wherein the second plurality of droplets is formed in a T-junction upstream of the first plurality of droplets.
   186. The method of any one of clauses 119-184, wherein the optical detector comprises a detection region.
   187. The method of any one of clauses 119-185, wherein the microfluidic device comprises a merger junction, wherein the one or more droplets of the first plurality of droplets is selectively merged with the one or more droplets of the second plurality of droplets by applying an electric field based on the detection of the first fluorescent moiety.
   188. The method of clause 186, wherein the detection region is upstream of the merger junction.
   189. The method of clause 186 or 187, wherein the one or more droplets of the first plurality of droplets pair with the one or more of the second plurality of reagent droplets in a region in the microfluidic device that is upstream of the merger junction.
   190. A microfluidic device comprising:
an optical detector for detecting an optical property of one or more droplets of a plurality of droplets;
an electrode; and
an automated system, wherein the automated system applies an electric field via the electrode to selectively merge the one or more droplets of a first plurality of droplets with one or more droplets of a second plurality of droplets based on the detection of the optical property.
   191. The device of clause 189, further comprising one or more droplet makers and one or more flow channels, wherein the one or more flow channels are fluidically connected to the one or more droplet makers and configured to receive one or more droplets therefrom.
   192. The device of clause 190, wherein the second plurality of droplets is formed upstream of the one or more droplets of the first plurality of droplets in the one or more flow channels.
   193. The device of clause 191, wherein the second plurality of droplets is formed in a T-junction upstream of the one or more droplets of the first plurality of droplets in the one or more flow channels.
   194. The device of any one of clauses 189-192, wherein the optical detector comprises a detection region.
   195. The device of any one of clauses 190-193, wherein the one or more flow channels comprise a merger junction, wherein the one or more droplets of the first plurality of droplets is selectively merged with the one or more droplets of the second plurality of droplets by activating the electrode based on the detection of an optical property.
   196. The device of clause 194, wherein the detection region is upstream of the merger junction.
   197. The device of any one of clauses 189-195, further comprising one or more output containers wherein the plurality of droplets is collected.
   198. The device of clause 196, wherein the one or more output containers are fluidically connected to one or more collection tubes comprising valves.
   199. The device of any one of clauses 189-197, wherein the microfluidic device does not comprise a sorter to physically sort the first plurality of droplets.
   200. The device of any one of clauses 189-197, wherein the microfluidic device further comprises a sorter to physically sort the first plurality of droplets.
   201. The device of clause 199, wherein the sorter physically separates the first plurality of droplets.
   202. A system comprising:
the microfluidic device of any one of clauses 189 to 200;
a power source; and
a controller, wherein the controller is configured to selectively enable or disable an electrical connection between the power source and the electrode thereby providing an active or inactive electrode respectively.
   203. A kit comprising one or more of the microfluidic devices of any one of clauses 189 to 200 and the system of clause 201.
   204. The kit of clause 202, further comprising instructions to carry out the methods of any one of clauses 1 to 188.

### EXAMPLES

Droplet microfluidics has significantly expanded the throughput and repertoire of single cell analysis by allowing soluble molecular biology reagents to be co-incubated with individual cells. In some embodiments, the instant disclosure provides a microfluidic system to target cell subsets with greatly reduced engineering complexity compared to droplet sorting. Instead of physically separating cells, the cells are tagged by selective reagent addition. Only droplets of interest receive reagents and therefore only droplets of interest are analyzed in downstream processing. Chemicals, small molecules, proteins, DNA, RNA, oligonucleotides, buffers, enzymes, beads, cells, or viruses can be selectively added to droplets of interest so that reactions (chemical synthesis, polymerase chain reactions, reverse transcription, transfection, transduction, transformation, etc.) occur only in those droplets. Selective merger can also be used to recover material without the need to physically sort by merging with probes or beads that can be later purified, or by triggering the formation of hydrogels that are easily recovered by filtration. Therefore, selective merger can replace sorting in many cases, but also adds the additional ability to perform reactions that aid downstream processing.

Addition of reagents to droplets is an important step in many droplet microfluidic workflows. Several methods are available to achieve this goal including pico-injection, triple emulsion coalescence, and droplet merger. These techniques indiscriminately add a defined volume to each droplet. Selective addition of reagents, demonstrated in the instant disclosure by the targeted merger of a smaller drop containing a detectable fluorescent signal with a larger drop containing reagents, can be used to analyze a subpopulation of droplets. Other methods, including selective pico-injection, and selective stream-merger are also possible. In many applications, selective addition of reagents enables enrichment without the need to physically sort. In some embodiments, both selective merger and sorting is carried out, wherein droplets are merged and sorted simultaneously. This removes unwanted material from downstream processing.

There are several important advantages of selective reagent addition for the isolation of sample subpopulations. First, the challenges of handling small cell numbers are avoided. Second, the complexity of droplet sorting is replaced with a significantly simpler device. Droplet sorting requires substantial expertise and as a result, commercial systems to perform it are not currently available. Third, fluorescent signals resulting from soluble assays or two-step droplet workflows can be selectively triggered, which expands the type of subpopulations that can be targeted. Workflows that require pre-incubation in droplets, lysis using proteases, or droplet culturing are now amenable to enrichment without sorting. Lastly, the technique can be implemented via any method that rapidly combines separate streams in an oil-based carrier phase, including droplet merger (22-24), pico-injection (25, 26), or stream merger, and is therefore compatible with many existing commercial and academic droplet workflows. Existing instruments for single cell DNA sequencing already incorporate droplet merger as an essential step in the barcoding workflow (27-30). A valuable attribute of the instant disclosure is thus that it can be integrated into existing merger devices without modification of microfluidic chips, which already have all the fluidic components necessary. Chip operation is also modified since, rather than the electrode always being on to merge all droplets, it is switched off and on to merge select droplets.

An important application of the instant disclosure is single cell sequencing. Many droplet microfluidic systems use barcode beads to obtain single cell resolution, but these devices barcode and sequence every cell loaded in a droplet. An important but rare subpopulation representing 1% of the total would mean 99% of the information generated is uninformative. The cost of sequencing is therefore distributed over a large population, instead of the cells of interest. Although additional handling or sorting of populations prior to encapsulation is feasible, this becomes prohibitive as the number of cells decreases due to difficulties in accurate counting and resuspension of limited inputs. Integrating enrichment into the barcoding step avoids these problems. Additionally, our approach can be applied to many populations in a sample—it is possible to selectively barcode subsets of interest sequentially. In this manner, and without sorting, it is possible to serially barcode subpopulations by diverting each to a different collection tube with a simple valve. In droplet microfluidic workflows where pre-sorting is impossible, as when soluble assays or growth phenotypes are performed in droplets, selective merger offers an attractive alternative to droplet sorting, and one that is easier to integrate into commercial systems that require robust and operator-free usage.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); and the like.

### Example 1: Efficient Targeting of Droplet Subpopulations Using Selective Merger

The instant disclosure provides a mechanism to selectively add reagents to droplets based on detection of a property of droplets (e.g., fluorescence, absorbance, etc.) and triggers the addition of reagents only to droplets of the desired type. This enables specific droplets to be targeted for downstream processing, thereby enriching subpopulations of interest.

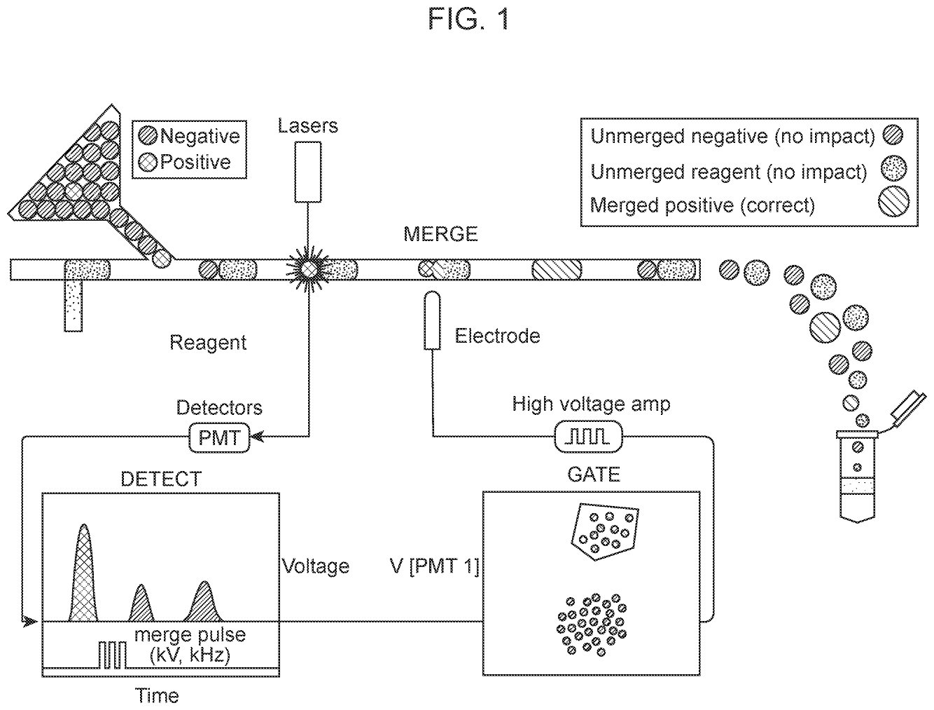

Droplet merger is an important component of many droplet microfluidic workflows because it allows the composition of each droplet to be precisely modified. This enables two-step workflows that utilize off chip temperature regulation, require sequential addition of incompatible chemicals, or precise temporal control of assay components. Merger has been used in numerous studies with microfluidics, including single cell genome sequencing (1, 2), single molecule haplotyping (17), and high throughput screening (18). This technique was exploited to target subpopulations by selectively adding reagents only to the desired drops. To perform selective merger, an emulsion comprising positive and negative fluorescent droplet populations is introduced into the device. These droplets are interdigitated with reagent droplets, and optically probed when passing through the detection region which sits just upstream of the merger device. Merging is triggered on droplets that fall within user-defined optical gates, and all droplets, whether merged or not, are collected in the output container (FIG. 1). The processed emulsion is incubated to allow reactions to occur in the merged droplets, and the emulsion is ruptured and reaction products recovered for analysis.

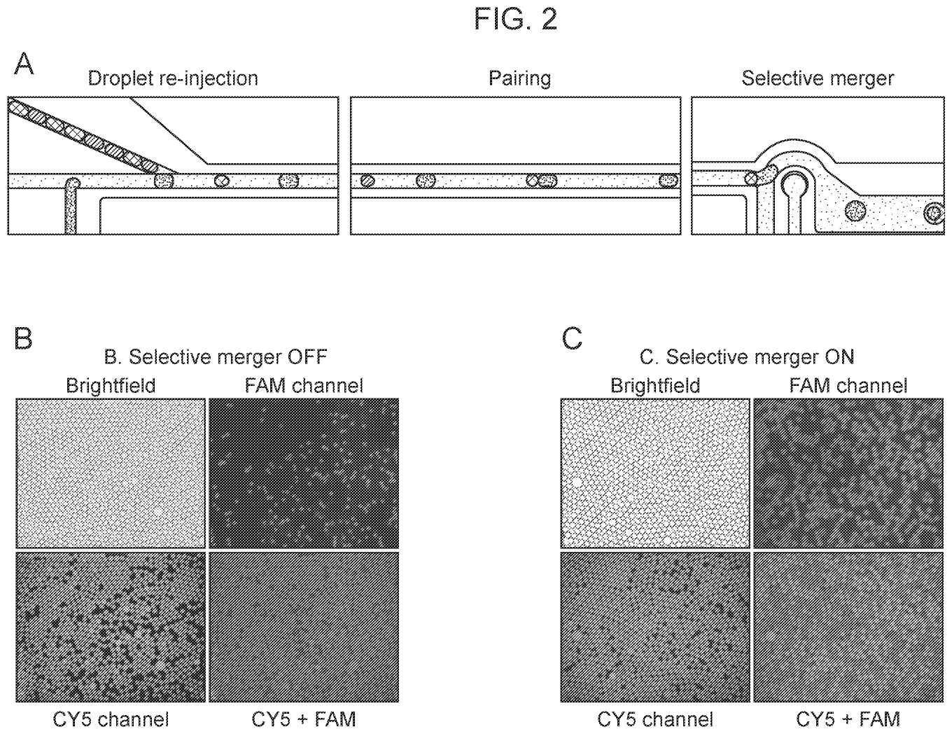

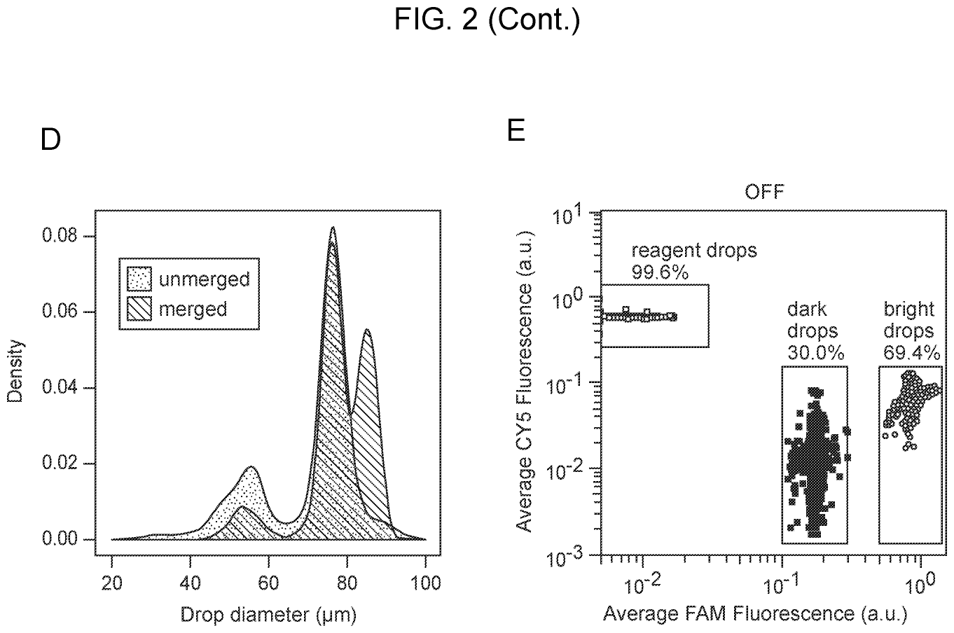

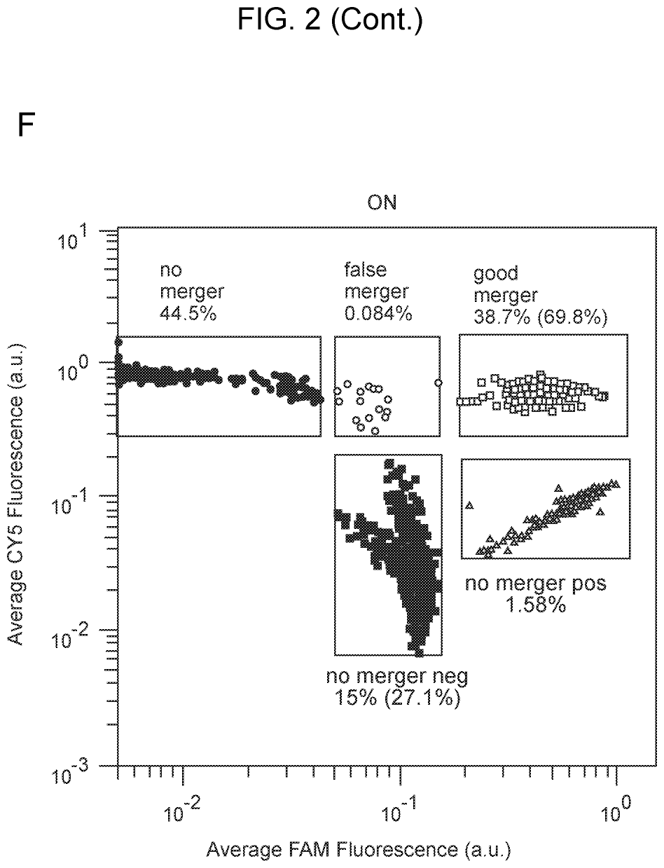

To characterize the efficiency of the microfluidic approach for selectively targeting droplet subpopulations, droplets containing two concentrations of a fluorescent dye (FAM+, FAM−) and target the positive population for merger were introduced. Reagent droplets (CY5+) are formed in a T-junction just upstream of the reinjected droplets (FIG. 2, Panel A, left), causing the droplets to pair due to size differences (FIG. 2, Panel A, middle). The paired droplets enter the merger junction in contact, allowing efficient coalescence when the electrode is on (FIG. 2, Panel A, right). With the electrode off, the paired droplets do not merge and thus instances of double positive (FAM+CY5+) are rare (FIG. 2, Panel B). When merger of positive droplets with reagent droplets is selectively triggered, the number double positives (FAM+CY5+) increases significantly (FIG. 2, Panel C), and the diameters the droplets shift from a population of two sizes (55 μm targets, 75 μm reagents) to a population of three sizes (55 μm targets, 75 μm reagents, 85 μm merged) (FIG. 2, Panel D). To quantitatively assess selective merger efficiency, droplet cytometry was used to measure droplet fluorescence for a large population. False merger (FAM− CY5+) occurs 0.085% of the time, and failed merger (FAM+CY5−) occurs 1.58% of the time (FIG. 2, Panel E). These results demonstrate the highly specific ability of fluorescence-activated droplet merger to add reagents to target droplet subpopulations.

### Example 2: Targeted Single Cell RNA-Sequencing of Selected Subpopulations

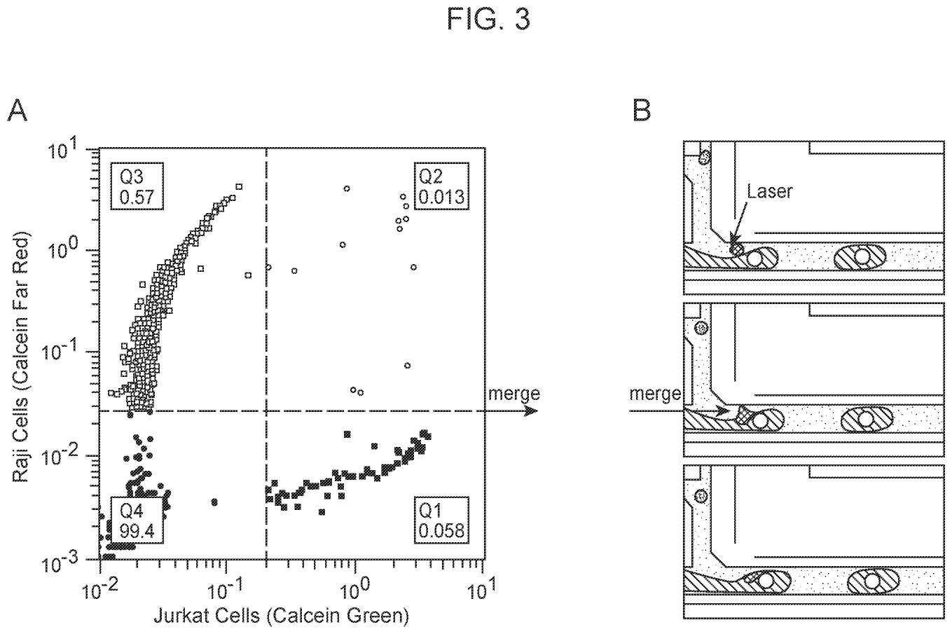

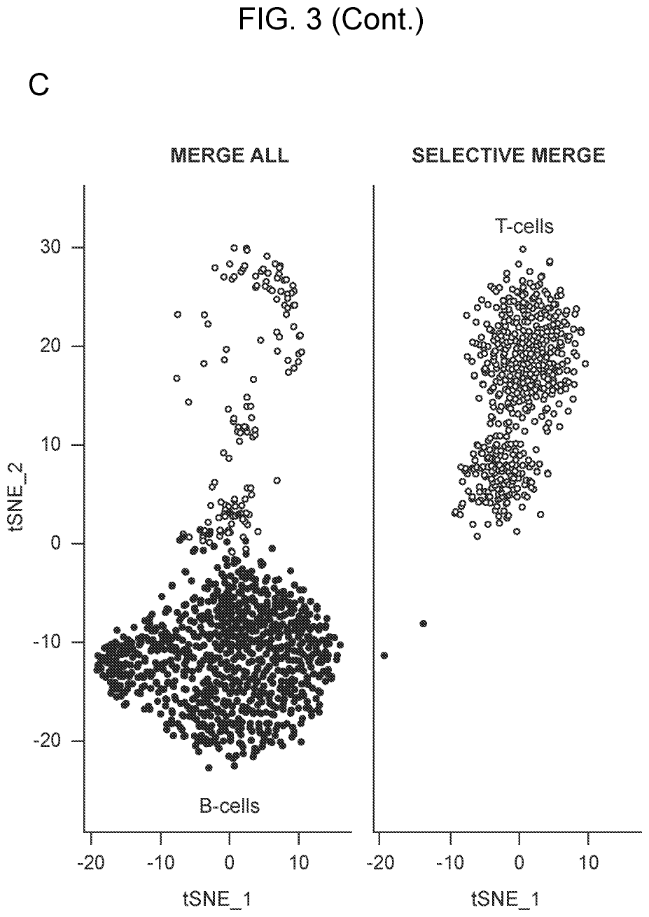

Single cell RNA sequencing is one of the broadest and most important contributions of droplet microfluidics to biology. It allows massive, heterogeneous populations of cells to be characterized at the single cell level rapidly and cost efficiently. However, existing methods cannot focus analysis on interesting subpopulations, resulting in significant waste of reagents and sequencing on uninteresting cells. Single cell sequencing approaches employ bead-based reactions to amplify and label cellular mRNAs with unique barcodes that enable in silico assignment of sequencing data to single cells. In such workflows, cells are paired with barcode beads, irrespective of identity, and the whole population is sequenced. Selective merger provides a simple way to sequence a subpopulation without having to pre-sort cells. To illustrate this, the approach to a mixed population of B-cells (Raji) and T-cells (Jurkat), stained separately so they can be identified by their fluorescence was applied (FIG. 3, Panel A). The B-cells are loaded at ten times the T-cells concentration and the resultant emulsion processed in a selective droplet merger device. For this device, the bead solution is sufficiently conductive to induce merger by direct electrification of the liquid stream (FIG. 3, Panel B). Merger is induced with a barcoded bead only when a droplet containing a T-cell (Calcein Green) is detected. When a Celcein Far Red cell passes through the detection window, the stream is unelectrified, resulting in no merging. All droplets are collected but reverse transcription with barcoded primers contained on the bead only occurs in merged drops. As a control, a sample with electrification always on was also collected, resulting in pairing of beads with all cells. Upon sequencing it was found that, as expected, merging all droplets results in single cells from both input populations: ˜90% B cells (Celcein Far Red) and ˜10% T cells (Calcein Green), as determined by single cell gene expression clustering (FIG. 3, Panel C, left). By contrast, when bead pairing is triggered only for T-cells, the vast majority (99.6%) classify correctly, (FIG. 3, Panel C, right). Misclassified cells (FIG. 3, Panel C), resulting from incorrect merger, are a small percentage (0.43%) and are still sequenced with single cell resolution. These results illustrate that droplet merger can selectively target specific cell populations for single cell RNA sequencing.

### Example 3: Targeted Single Cell DNA-Sequencing of Mutation Hotspots from Leukemia Cells

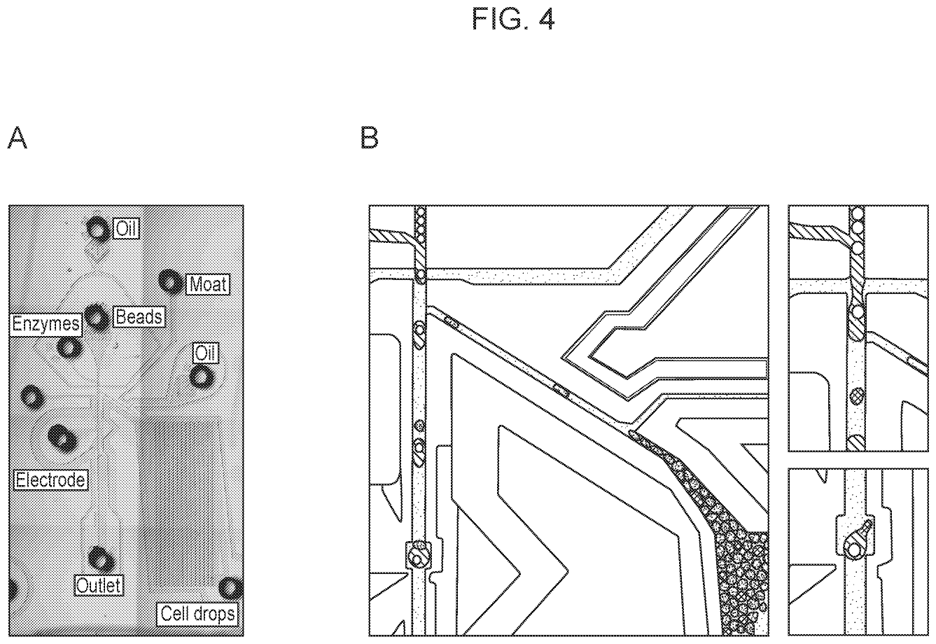

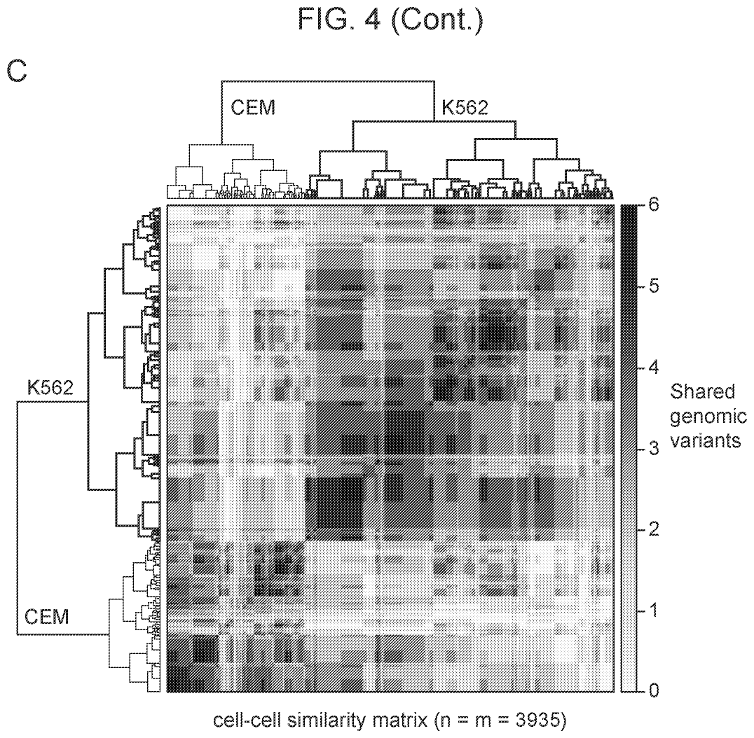

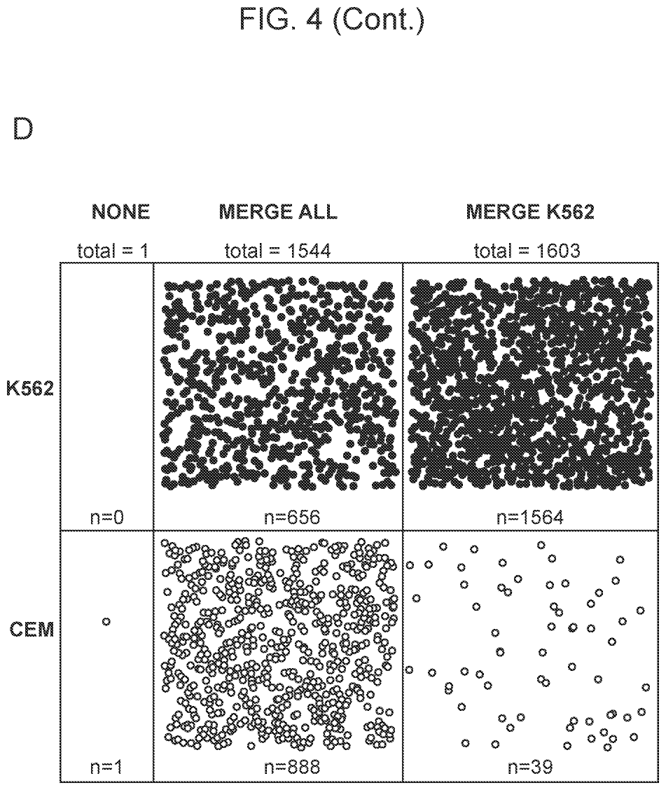

Droplet microfluidic technologies are enabling population scale single cell DNA sequencing with unprecedented throughput and cost efficiency (19-21). These technologies allow generation of rich and detailed genomic maps that relate clonal lineages and track the genomic programing of therapeutic resistance. Droplet merger is an essential step in these workflows because it allows cellular lysis and targeted PCR, two incompatible steps, to be performed sequentially. By separating lysis from PCR amplification of single cell DNA, these workflows achieve efficient completion of both steps. Because cell lysate is merged with beads to enable target gene amplification and barcoding, methods for single cell DNA-seq can be readily modified to analyze specific cell subsets by incorporating selective merger. To illustrate this, leukemia cells were targeted for single cell genome sequencing of a tumor hotspot panel containing 16 loci. Two cancer cell lines were stained— CEM (acute lymphoblastic leukemia) cells and K562 (bone marrow derived lymphoblast) cells—and mixed at a 1:1 ratio. The mixed cell suspension was co-flowed with lysis buffer and Cascade Blue dye; this dye acts as a droplet tag that allows us to detect all droplets, including ones devoid of cells, which aids in population gating. After lysis incubation, the cell droplets are introduced into the selective merger device, which adds the amplification mastermix, barcode beads, and genomic hotspot PCR primers by droplet merger (FIG. 4, Panels A and B).

Three libraries were prepared: K562 cells (merged high green), merge all drops (merge Cascade blue), and merge none (electrode off), in individual tubes with identical PCR conditions and load equal amounts from each library on the sequencer. Approximately 1,600 cells were sequenced per selective-merger condition, close to the expected 2,000 cells. Cells were classified based on known genomic mutations in each of the cell lines (FIG. 4, Panel C). As expected, when all cells are merged with reagents, equal proportions of each cell type were recovered (FIG. 4, Panel D). For the selective merger runs, 39/1564 (2.5%) contamination was observed, which corresponds to the expected doublet rate for the experiment. This can be further reduced if a second dye is used to exclude doublets, which would also allow for higher loading of cells per drops and increased throughput. Based on these results, with the current microfluidic design, enrichment of cells with rarity of 1 in 1,000 is possible. This result illustrates the ease with which cell subsets can be targeted for analysis on existing commercial workflows for single cell DNA sequencing. Focusing all sequencing power on subpopulations of most interest will allow deeper and more cost-efficient recovery of important and actionable information

### Example 4: Selective Merger and Sorting

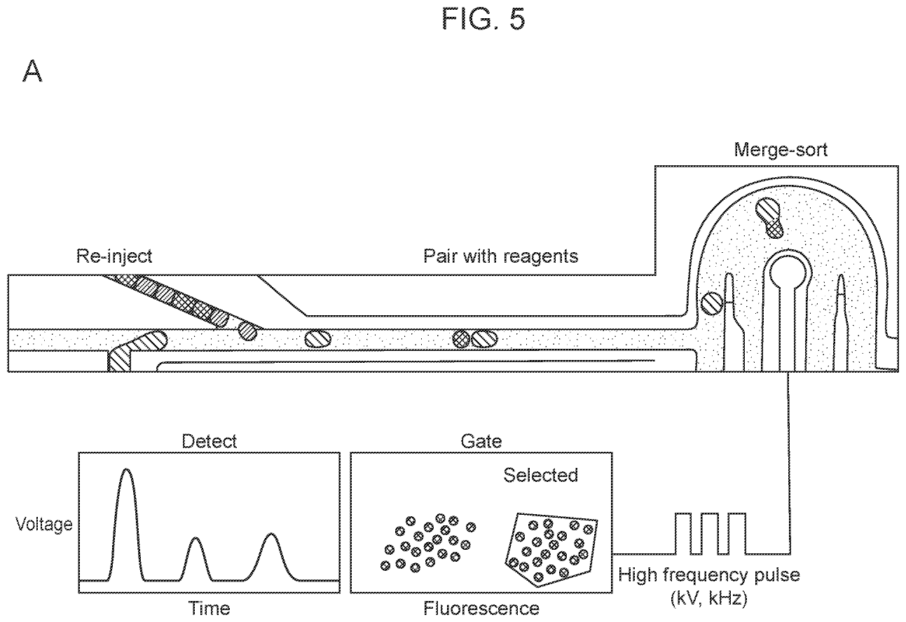

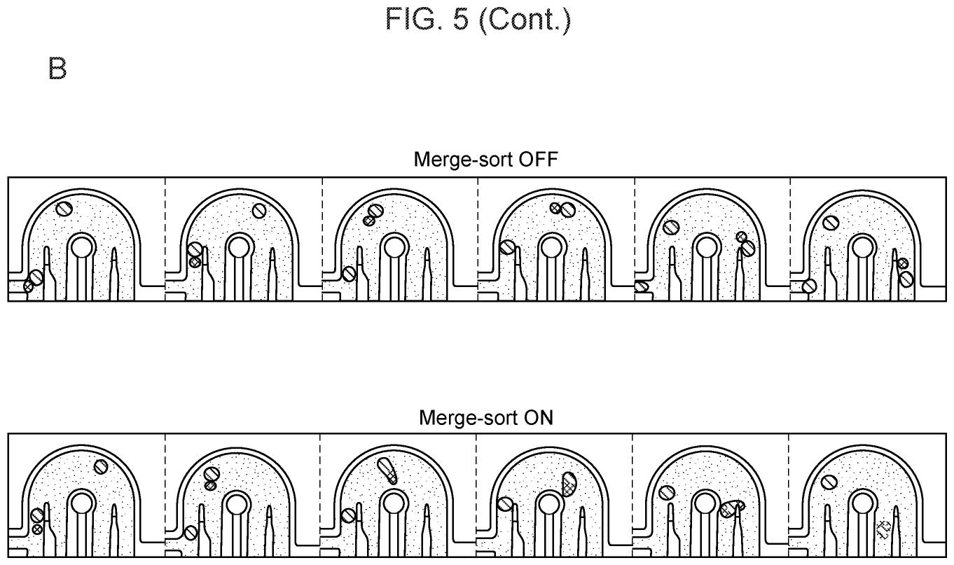

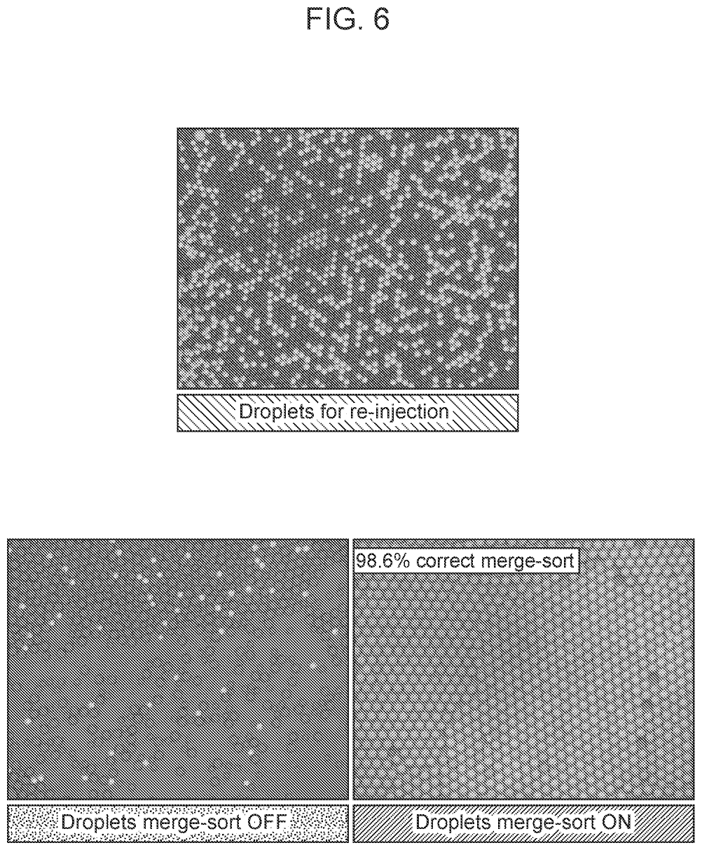

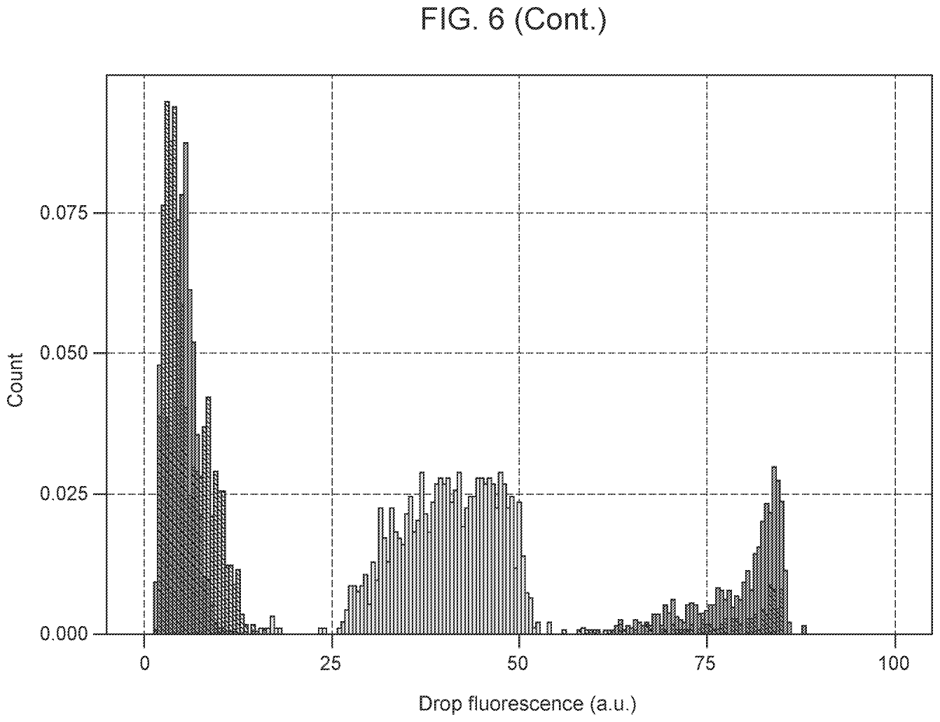

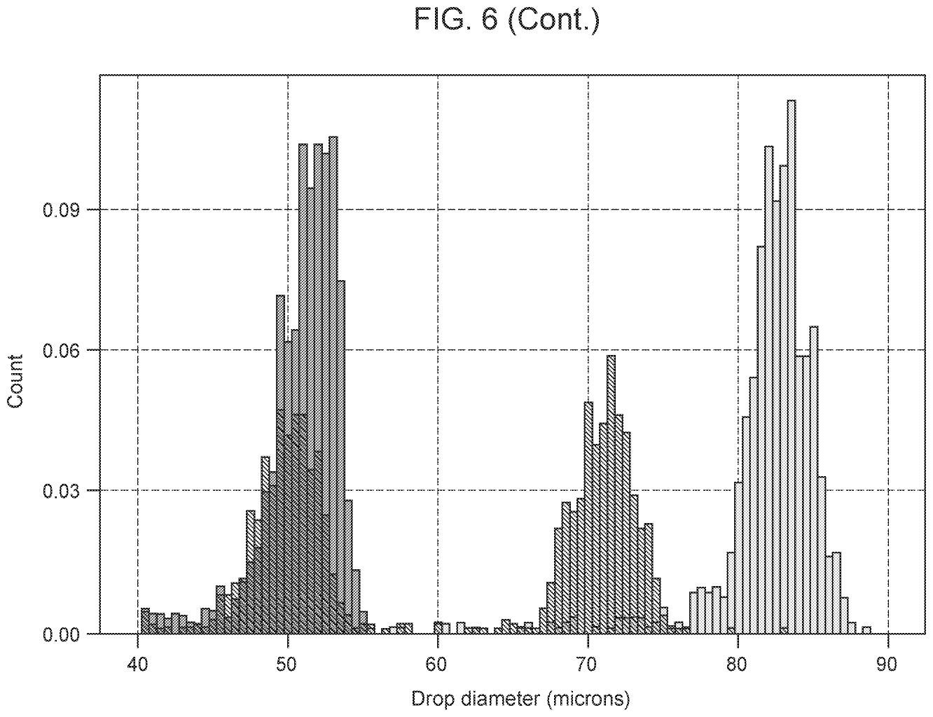

Selective merger and sorting can also be achieved using the instant methods and devices. In some embodiments, droplets are merged and physically sorted simultaneously (FIG. 5, Panels A and B and FIG. 6). This removes unwanted material from downstream processing. This is particularly important for some sequencing applications. For example, selective merger of Phi29 and reagents for multiple displacement amplification can be used to selectively amplify cells or molecules of interest. However, if the sorting step is not implemented, significant material from untargeted cells can end up in downstream processing. For example, by adding simultaneous merge-sort, multiple displacement amplification (MDA) reagents are added to selected droplets and separated, allowing digital droplet MDA (ddMDA) on subpopulations without contamination.

The typical method to enrich cells or molecules is through physical separation. Provided herein is an alternative approach that selectively tags droplet contents and allows analysis of their contents without physical separation. Performing targeted reactions is equivalent to sorting in many cases. It is particularly useful for concentrating sequencing power on cells or molecules of interest and is compatible with bead-based barcoding techniques. The instant methods, devices and systems can be applied more widely than droplet sorting, especially in systems that require enhanced levels of reliability, for example, to recover or analyse any material that can be compartmentalized and detected in droplets.

### Materials and Methods

### Microfluidic Fabrication and Device Construction

Three-inch silicon wafers are spin coated with SU-8 2025 photoresist (MicroChem, Westborough, Mass., USA), and UV-patterned using a mask aligner (SUSS MJB3). PDMS prepolymer and curing agent (Momentive, Waterford, N.Y., USA; RTV 615) are mixed vigorously at a 10 to 1 ratio, degassed in a vacuum chamber, and poured onto the master mold. The mold is degassed and baked at 65° C. overnight before being removed and punched with a 0.75-mm biopsy punch (Ted Pella, Inc., Redding, Calif., USA; Harris Uni-Core 0.75). The PDMS replica and a glass slide (75×50×1.0 mm, 12-550 C, Fisher Scientific) are plasma treated (Technics Plasma etcher 500-II) and bonded. The complete device is placed at 150° C. to strengthen bonds, and further baked overnight. The device is treated with Aquapel for five-minutes, purged with air, flushed with oil (Fluorinert FC-40), purged with air, and baked for 30 minutes before use.

### Dye-Based Validation of Selective Merger

A FAM labeled oligonucleotide in PBS is used to generate 45 μm droplets using a bubble-triggered droplet generator running 2% ionic Krytox, prepared as previously described (19). Two concentrations (1 μM and 0.1 uM) of FAM droplets are produced and mixed. Drops are re-injected and paired with 80 μm droplets generated on-chip that contain BSA-CY5 (1 μM) in PBS. Selective merger of paired droplets is achieved by triggering a salt-water electrode (2M NaCl) in response to fluorescence. Analysis of droplet fluorescence is performed on a droplet cytometer built as previously described (9). The system contains three lasers (473 nm, 532 nm, 638 nm) for excitation and filter sets to direct fluorescence to three photomultiplier tubes (PMM01, Thorlabs). Droplet fluorescence values are recorded, exported, and analyzed in FlowJo.

### Single Cell Transcriptomics

Raji (ATCC CCL-86) and Jurkat (ATCC CRL-2901) cells were cultured with Gibco RPMI Media 1640 (ThermoFisher 11875093) containing 10% Fetal Bovine Serum with Penicillin and Streptomycin (Thermo Fisher 15140122). Cells are stained with CellTrace Far Red (Thermofisher #C34564) and CellTrace Calcein green (Thermofisher #C34852), respectively. Cells are counted and mixed at a ratio of 10:1 Raji:Jurkat. Cell droplets, generated on-chip, are selectively merged with a liquid stream containing reagents for reverse transcription and barcoded beads (inDrops v3, Harvard Single Cell Core), according to establish recipes and protocols (3, 31). Sequencing reads are processed using the inDrops pipeline available on github (https://github.com/indrops/indrops) to generate a table of counts per genes per cell. t-Distributed Stochastic Neighbor Embedding (t-SNE) is used to cluster and visualize single cell RNA-seq data (32).

### Single Cell Genomics

Chronic myelogenous leukemia cells (K-562 ATCC CCL-243) and acute lymphoblastic leukemia cells (CEM/C1 ATCC CRL-2265) are cultured with Gibco RPMI Media 1640 (ThermoFisher #11875093) containing 10% Fetal Bovine Serum with Penicillin and Streptomycin (Thermo Fisher #15140122). Calcein (25 μM Calcein, ThermoFisher) is used to stain cells by incubating on ice for 30 min in 1×PBS. After staining, cells are washed twice (HBSS, no calcium, no magnesium, 14170112, ThermoFisher) and re-suspended in HBSS containing 18% OptiPrep Density Gradient Medium (Sigma-Aldrich). CEM cells are stained with Calcein Red-Orange (Thermofisher #C34851) and K562 cells are stained with Calcein Green AM (Thermofisher #C34852). Cells are counted, re-suspended to 3M cells/ml, mixed at a ratio of 1:1, and co-flowed with Mission Bio lysis buffer to generate 45 μm droplets. Cascade Blue was included as a drop dye to enable triggering and merging of all drops as an experimental control. Custom barcode beads were generated targeting 16 amplicons of the Mission Bio Acute Myeloid Leukemia panel. These amplicons are chosen because K562 and CEM have different SNPs in those genomic locations that allow for their subsequent identification. Library preparation and sequencing is performed according to Mission Bio's protocol.

Each cell was genotyped by demultiplexing the sequencing reads by cell barcodes and variant calling all amplicons. 1100 called variants in the cell pool were reduced to the most informative polymorphisms to display a high fraction of cells with alternate homozygous or heterozygous calls and low drop-out rate, giving rise to the following short list (chr7:148504716: AG/A, chr17:7578115: T/C, chr13:28602226: AAGAG/A, chr6:17076740: T/C, chr13:28602227: AGAG/*, chr13:28602229: AGAGAGAG/*, chr5:170837457: A/G, chr16:8569722: T/C). A cell-cell similarity matrix was constructed by performing the dot product between the 6-dimensional feature vectors of each cell pair (bold faced above, one-hot encoded). This is, in principle, a non-normalized variant of the Jaccard index. The resulting similarity matrix was clustered with Ward's minimum variance method (FIG. 4, Panel B). The two top tier clusters represent the two cell lines K562 and CEM, which is confirmed by splitting all cells into the assigned clusters and plotting versus the library index (FIG. 4, Panel C).

Although the foregoing invention has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

### REFERENCES

1. F. Lan, B. Demaree, N. Ahmed, A. R. Abate, Single-cell genome sequencing at ultra-high-throughput with microfluidic droplet barcoding. Nat Biotechnol 35, 640-646 (2017).
2. B. Demaree, D. Weisgerber, F. Lan, A. R. Abate, An Ultrahigh-throughput Microfluidic Platform for Single-cell Genome Sequencing. J Vis Exp 10.3791/57598 (2018).
3. A. M. Klein et al., Droplet Barcoding for Single-Cell Transcriptomics Applied to Embryonic Stem Cells. Cell 161, 1187-1201 (2015).
4. E. Z. Macosko et al., Highly Parallel Genome-wide Expression Profiling of Individual Cells Using Nanoliter Droplets. Cell 161, 1202-1214 (2015).
5. P. Shahi, S. C. Kim, J. R. Haliburton, Z. J. Gartner, A. R. Abate, Abseq: Ultrahigh-throughput single cell protein profiling with droplet microfluidic barcoding. Sci Rep 7, 44447 (2017).
6. M. Stoeckius et al., Simultaneous epitope and transcriptome measurement in single cells. Nat Methods 14, 865-868 (2017).
7. D. E. Wagner et al., Single-cell mapping of gene expression landscapes and lineage in the zebrafish embryo. Science 360, 981-987 (2018).
8. R. Zilionis et al., Single-Cell Transcriptomics of Human and Mouse Lung Cancers Reveals Conserved Myeloid Populations across Individuals and Species. Immunity 50, 1317-1334 e1310 (2019).
9. M. A. Wheeler et al., MAFG-driven astrocytes promote CNS inflammation. Nature 10.1038/s41586-020-1999-0 (2020).
10. A. Dixit et al., Perturb-Seq: Dissecting Molecular Circuits with Scalable Single-Cell RNA Profiling of Pooled Genetic Screens. Cell 167, 1853-1866 e1817 (2016).
11. B. Adamson et al., A Multiplexed Single-Cell CRISPR Screening Platform Enables Systematic Dissection of the Unfolded Protein Response. Cell 167, 1867-1882 e1821 (2016).
12. K. Ahn et al., Dielectrophoretic manipulation of drops for high-speed microfluidic sorting devices. Applied Physics Letters 88, 024104-024103 (2006).
13. J.-C. Baret et al., Fluorescence-activated droplet sorting (FADS): efficient microfluidic cell sorting based on enzymatic activity. Lab on a Chip 9, 1850-1810 (2009).
14. T. Franke, A. R. Abate, D. A. Weitz, A. Wixforth, Surface acoustic wave (SAW) directed droplet flow in microfluidics for PDMS devices. Lab on a Chip 9, 2625-2627 (2009).
15. T. Franke, S. Braunmüller, L. Schmid, A. Wixforth, D. A. Weitz, Surface acoustic wave actuated cell sorting (SAWACS). Lab on a Chip 10, 789-794 (2010).
16. L. Mazutis et al., Single-cell analysis and sorting using droplet-based microfluidics. Nature Protocols 8, 870-891 (2013).
17. F. Lan, J. R. Haliburton, A. Yuan, A. R. Abate, Droplet barcoding for massively parallel single-molecule deep sequencing. Nature Communications 7, 11784 (2016).
18. E. Brouzes et al., Droplet microfluidic technology for single-cell high-throughput screening. Proceedings of the National Academy of Sciences of the United States of America 106, 14195-14200 (2009).
19. M. Pellegrino et al., High-throughput single-cell DNA sequencing of acute myeloid leukemia tumors with droplet microfluidics. Genome Res 28, 1345-1352 (2018).
20. L. Xu et al., Clonal Evolution and Changes in Two AML Patients Detected with A Novel Single-Cell DNA Sequencing Platform. Sci Rep 9, 11119 (2019).
21. C. M. McMahon et al., Clonal Selection with RAS Pathway Activation Mediates Secondary Clinical Resistance to Selective FLT3 Inhibition in Acute Myeloid Leukemia. Cancer Discov 9, 1050-1063 (2019).
22. L. Mazutis, J. C. Baret, A. D. Griffiths, A fast and efficient microfluidic system for highly selective one-to-one droplet fusion. Lab Chip 9, 2665-2672 (2009).
23. X. Niu, S. Gulati, J. B. Edel, A. J. deMello, Pillar-induced droplet merging in microfluidic circuits. Lab Chip 8, 1837-1841 (2008).
24. I. Akartuna, D. M. Aubrecht, T. E. Kodger, D. A. Weitz, Chemically induced coalescence in droplet-based microfluidics. Lab Chip 15, 1140-1144 (2015).
25. D. J. Eastburn, A. Sciambi, A. R. Abate, Picoinjection enables digital detection of RNA with droplet rt-PCR. PloS one 10.1371/journal.pone.0062961.g001 (2013).
26. A. R. Abate, T. Hung, P. Mary, J. J. Agresti, D. A. Weitz, High-throughput injection with microfluidics using picoinjectors. Proceedings of the National Academy of Sciences of the United States of America 107, 19163-19166 (2010).
27. D. J. Eastburn, A. Sciambi, A. R. Abate, Identification and genetic analysis of cancer cells with PCR-activated cell sorting. Nucleic Acids Research 42, e128 (2014).
28. S. W. Lim, S. T. Lance, K. M. Stedman, A. R. Abate, PCR-activated cell sorting as a general, cultivation-free method for high-throughput identification and enrichment of virus hosts. Journal of Virological Methods 242, 14-21 (2017).
29. S. W. Lim, T. M. Tran, A. R. Abate, PCR-Activated Cell Sorting for Cultivation-Free Enrichment and Sequencing of Rare Microbes. PloS one 10, e0113549-0113516 (2015).
30. M. Pellegrino et al., RNA-Seq following PCR-based sorting reveals rare cell transcriptional signatures. BMC Genomics 17, 361 (2016).
31. R. Zilionis et al., Single-cell barcoding and sequencing using droplet microfluidics. Nature Methods 12, 44-73 (2016).
32. L. v. d. M. a. G. Hinton, Visualizing High-Dimensional Data Using t-SNE. Journal of Machine Learning Research 9, 2579-2605 (2008).

What is claimed is:

1. A method of selectively adding one or more reagents to one or more target molecules comprising:
flowing an emulsion comprising a plurality of droplets through a microfluidic device, wherein at least one droplet of the plurality of droplets comprises a target molecule;
flowing a plurality of reagent droplets comprising one or more reagents through the microfluidic device;
detecting a property of one or more droplets of the plurality of droplets; and
selectively merging one or more droplets of the plurality of droplets with one or more droplets of the plurality of reagent droplets based on the detection of the property.

2. The method of claim 1, wherein the property is an optical property.

3. The method of claim 2, wherein the single cell is labelled with a first fluorescent moiety and the optical property is fluorescence of the first fluorescent moiety.

4. The method of claim 2, wherein the optical property is absorbance.

5. The method of claim 1, wherein the property is size.

6. The method of claim 1, wherein the property is conductivity.

7. The method of any one of claims 1-6, wherein the selective merging comprises applying an electric field to selectively merge the one or more droplets with one or more droplets of the plurality of reagent droplets.

8. The method of any one of claims 1-6, wherein the selective merging comprises stream merging of the one or more droplets with one or more droplets of the plurality of reagent droplets.

9. The method of any one of claims 1-6, wherein the selective merging comprises pico-injection.

10. The method of any one of claims 1-6, wherein the selective merging comprises triple-emulsion coalescence.

11. The method of any one of claims 1-9, wherein the one or more reagents is a nucleic acid.

12. The method of claim 10, wherein the nucleic acid is a ribonucleic acid (RNA).

13. The method of claim 10, wherein the nucleic acid is a deoxyribonucleic acid (DNA).

14. The method of claim 12, wherein the DNA is an oligonucleotide.

15. The method of claim 12, wherein the DNA is plasmid DNA.

16. The method of any one of claims 1-9, wherein the one or more reagents is a protein.

17. The method of any one of claims 1-9, wherein the one or more reagents is a peptide.

18. The method of any one of claims 1-9, wherein the one or more reagents is a buffer.

19. The method of any one of claims 1-9, wherein the one or more reagents is an enzyme.

20. The method of claim 18, wherein the enzyme is a transposase enzyme.

21. The method of any one of claims 1-9, wherein the one or more reagents is a bead.

22. The method of claim 20, wherein the bead is a barcoded bead.

23. The method of any one of claims 1-9, wherein the one or more reagents is an amplification mastermix.

24. The method of any one of claims 1-9, wherein the one or more reagents is a polymerase chain reaction (PCR) primer.

25. The method of any one of claims 1-9, wherein the one or more reagents is a multiple displacement amplification (MDA) reagent.

26. The method of any one of claims 1-9, wherein the one or more reagents is a cell.

27. The method of any one of claims 1-9, wherein the one or more reagents is a microbe.

28. The method of claim 26, wherein the microbe is a virus.

29. The method of claim 26, wherein the microbe is a fungus.

30. The method of claim 26, wherein the microbe is a bacterium.

31. The method of any one of claims 1-9, wherein the one or more reagents is a chemical.

32. The method of claim 30, wherein the chemical is a small molecule.

33. The method of claim 30, wherein the chemical is a hydrogel reversing agent.

34. The method of claim 30, wherein the chemical is a hydrogel precursor.

35. The method of claim 31, wherein the hydrogel precursor is tetramethylethylenediamine (TEMED).

36. The method of claim 31, wherein the hydrogel precursor is acrylamide.

37. The method of claim 31, wherein the hydrogel precursor is a calcium solution.

38. The method of claim 31, wherein the hydrogel precursor is a polyethylene glycol (PEG) diacrylate solution.

39. The method of any one of claims 1-37, wherein the one or more droplets of the plurality of droplets comprises a cell.

40. The method of claim 38, wherein each droplet of the plurality of droplets comprises not more than one cell.

41. The method of claim 38 or 39, wherein the target molecule is a rare cell.

42. The method of claim 40, wherein the rare cell is a cancer cell.

43. A method of single-cell sequencing comprising selectively adding one or more reagents to one or more target cells, the method comprising:
flowing an emulsion comprising a plurality of droplets through a microfluidic device, wherein at least one droplet of the plurality of droplets comprises a single cell;
flowing a plurality of reagent droplets comprising one or more reagents through the microfluidic device;
detecting a property of one or more droplets of the plurality of droplets;
applying an electric field to selectively merge one or more droplets of the plurality of droplets with one or more droplets of the plurality of reagent droplets based on the detection of the property; and
sequencing the selectively merged one or more droplets of the plurality of droplets.

44. The method of claim 42, wherein the property is an optical property.

45. The method of claim 43, wherein the single cell is labelled with a first fluorescent moiety and the optical property is fluorescence of the first fluorescent moiety.

46. The method of claim 43, wherein the optical property is absorbance.

47. The method of claim 42, wherein the property is size.

48. The method of claim 42, wherein the property is conductivity.

49. The method of any one of claims 42-47, wherein the selective merging comprises applying an electric field to selectively merge the one or more droplets with one or more droplets of the plurality of reagent droplets.

50. The method of any one of claims 42-47, wherein the selective merging comprises stream merging of the one or more droplets with one or more droplets of the plurality of reagent droplets.

51. The method of any one of claims 42-47, wherein the selective merging comprises pico-injection.

52. The method of any one of claims 42-50, wherein the method comprises single-cell RNA sequencing.

53. The method of any one of claims 42-51, wherein the single cell labeled with a first fluorescent moiety is a rare cell.

54. The method of claim 52, wherein the rare cell is a cancer cell.

55. A method of hydrogel formation comprising selectively adding one or more reagents to one or more target cells, wherein the one or more reagents comprise a hydrogel precursor, the method comprising:
flowing an emulsion comprising a plurality of droplets through a microfluidic device, wherein at least one droplet of the plurality of droplets comprises a target cell;
flowing a plurality of reagent droplets comprising the one or more reagents through the microfluidic device;
detecting a property of one or more droplets of the plurality of droplets; and
selectively merging one or more droplets of the plurality of droplets with one or more droplets of the plurality of reagent droplets based on the detection of the property to form the hydrogel within said one or more droplets of the plurality of droplets.

56. The method of claim 54, wherein the property is an optical property.

57. The method of claim 55, wherein the single cell is labelled with a first fluorescent moiety and the optical property is fluorescence of the first fluorescent moiety.

58. The method of claim 55, wherein the optical property is absorbance.

59. The method of claim 54, wherein the property is size.

60. The method of claim 54, wherein the property is conductivity.

61. The method of any one of claims 54-59, wherein the selective merging comprises applying an electric field to selectively merge the one or more droplets with one or more droplets of the plurality of reagent droplets.

62. The method of any one of claims 54-59, wherein the selective merging comprises stream merging of the one or more droplets with one or more droplets of the plurality of reagent droplets.

63. The method of any one of claims 54-59, wherein the selective merging comprises pico-injection.

64. The method of any one of claims 54-62, wherein a polyacrylamide hydrogel is formed and wherein at least one droplet of the plurality of droplets comprises a target cell labeled with a first fluorescent moiety and ammonium persulfate and wherein the hydrogel precursors are tetramethylethylenediamine (TEMED) and acrylamide.

65. The method of any one of claims 54-63, wherein an alginate hydrogel is formed and wherein at least one droplet of the plurality of droplets comprises a target cell labeled with a first fluorescent moiety and alginate and wherein the hydrogel precursor is a calcium solution.

66. The method of any one of claims 54-64, wherein a PEG hydrogel is formed and wherein at least one droplet of the plurality of droplets comprises a target cell labeled with a first fluorescent moiety and PEG-Thiol and wherein the hydrogel precursor is a PEG diacrylate solution.

67. The method of any one of claims 54-65, wherein the target cell labeled with a first fluorescent moiety is a rare cell.

68. The method of claim 66, wherein the rare cell is a cancer cell.

69. The method of any one of claims 54-67, wherein the target cell labeled with a first fluorescent moiety is targeted for removal downstream.

70. A method of hydrogel dissolution by selectively adding one or more reagents to one or more target cells in a reversible hydrogel, wherein the one or more reagents comprise a hydrogel reversing agent, the method comprising:
flowing an emulsion comprising a plurality of droplets through a microfluidic device, wherein at least one droplet of the plurality of droplets comprises a target cell in a reversible hydrogel;
flowing a plurality of reagent droplets comprising the one or more reagents through the microfluidic device;
detecting a property of one or more droplets of the plurality of droplets; and
selectively merging one or more droplets of the plurality of droplets with one or more droplets of the plurality of reagent droplets based on the detection of the property to dissolve the hydrogel within said one or more droplets of the plurality of droplets.

71. The method of claim 69, wherein the property is an optical property.

72. The method of claim 70, wherein the single cell is labelled with a first fluorescent moiety and the optical property is fluorescence of the first fluorescent moiety.

73. The method of claim 70, wherein the optical property is absorbance.

74. The method of claim 69, wherein the property is size.

75. The method of claim 69, wherein the property is conductivity.

76. The method of any one of claims 69-74, wherein the selective merging comprises applying an electric field to selectively merge the one or more droplets with one or more droplets of the plurality of reagent droplets.

77. The method of any one of claims 69-74, wherein the selective merging comprises stream merging of the one or more droplets with one or more droplets of the plurality of reagent droplets.

78. The method of any one of claims 69-74, wherein the selective merging comprises pico-injection.

79. The method of any one of claims 69-77, wherein the reversible hydrogel is an alginate hydrogel with a cleavable crosslinker.

80. The method of any one of claims 69-78, wherein the reversible hydrogel is a PEG hydrogel with a cleavable crosslinker.

81. The method of any one of claims 69-79, wherein the reversible hydrogel is a polyacrylamide hydrogel with a cleavable crosslinker.

82. The method of any one of claims 69-80, wherein the hydrogel reversing agent is ethylenediaminetetraacetic acid (EDTA).

83. The method of any one of claims 69-80, wherein the hydrogel reversing agent is dithiothreitol (DTT).

84. The method of any one of claims 69-80, wherein the hydrogel reversing agent is ultraviolet (UV) light.

85. The method of any one of claims 69-83, wherein the target cell labeled with a first fluorescent moiety is targeted for removal downstream.

86. The method of any one of claims 1-84, wherein at least one droplet of the plurality of droplets comprises a non-target molecule labeled with a second fluorescent moiety that is distinct from the first fluorescent moiety.

87. The method of claim 85, wherein the at least one droplet of the plurality of droplets comprising the non-target molecule labeled with a second fluorescent moiety is not merged with one or more droplets of the plurality of reagent droplets based on the detection of the second fluorescent moiety.

88. The method of claim any one of claims 1-84, wherein at least one droplet of the plurality of droplets comprises a non-target molecule that is not labeled with a fluorescent moiety.

89. The method of claim 87, wherein the at least one droplet of the plurality of droplets comprising the non-target molecule not labeled with a fluorescent moiety is not merged with one or more droplets of the plurality of reagent droplets.

90. The method of claim 86 or 88, wherein the unmerged droplets are removed downstream.

91. The method of claim 86 or 88, wherein the unmerged droplets are recovered.

92. The method of claim 90, wherein the recovered unmerged droplets are recycled such that the method of any one of claims 1-88 is repeated with the recovered droplets.

93. The method of claim 91, wherein the recovered droplets are continuously recycled during performance of the method.

94. The method of any one of claims 1-92, wherein applying the electric field selectively merges the one or more droplets of the plurality of droplets comprising the target molecule labeled with a first fluorescent moiety with one or more droplets of the plurality of reagent droplets based on the detection of the first fluorescent moiety.

95. The method of any one of claim 86, 88 or 93, wherein the plurality of droplets is collected in one or more output containers.

96. The method of claim 94, wherein the plurality of droplets is collected in one or more output containers via one or more collection tubes comprising valves.

97. The method of claim 94 or 95, further comprising incubating the collected plurality of droplets to allow reactions to occur in the one or more droplets of the plurality of droplets merged with the one or more droplets of the plurality of reagent droplets to produce one or more reaction products.

98. The method of claim 96, wherein the reaction is a chemical synthesis reaction.

99. The method of claim 96, wherein the reaction comprises a polymerase chain reaction (PCR).

100. The method of claim 96, wherein the reaction comprises a multiple displacement amplification (MDA).

101. The method of claim 96, wherein the reaction comprises a reverse transcription reaction.

102. The method of claim 96, wherein the reaction comprises a transfection reaction.

103. The method of claim 96, wherein the reaction comprises a transduction reaction.

104. The method of claim 96, wherein the reaction comprises a transformation reaction.

105. The method of any one of claims 95-103, further comprising rupturing the plurality of droplets and recovering the reaction products for analysis.

106. The method of claim 104, wherein the reaction products are recovered via filtration.

107. The method of any one of claims 1-105, wherein the method does not comprise physically sorting the plurality of droplets via a sorter.

108. The method of any one of claims 1-105, wherein the microfluidic device comprises a sorter, and wherein the method further comprises physically sorting the plurality of droplets via the sorter.

109. The method of claim 107, wherein the sorting of the plurality of droplets is performed simultaneously with selectively merging the one or more droplets of the plurality of droplets based on the detection of the first fluorescent moiety.

110. The method of claim 107 or 108, wherein the sorting comprises physical separation of the plurality of droplets.

111. The method of any one of claims 107-109, wherein the one or more reagents is a multiple displacement amplification (MDA) reagent.

112. The method of any one of claims 1-110, wherein the optical detector comprises an optical fiber configured to apply excitation energy to one or more droplets of the plurality of droplets.

113. The method of claim 111, wherein the optical fiber is configured to collect a signal produced by the application of the excitation energy to one or more droplets of the plurality of droplets.

114. The method of any one of claims 1-112, wherein the plurality of reagent droplets are formed upstream of the plurality of droplets in the microfluidic device.

115. The method of claim 113, wherein the plurality of reagent droplets are formed in a T-junction upstream of the plurality of droplets.

116. The method of any one of claims 1-114, wherein the optical detector comprises a detection region.

117. The method of any one of claims 1-115, wherein the microfluidic device comprises a merger junction, wherein the one or more droplets of the plurality of droplets is selectively merged with the one or more droplets of the plurality of reagent droplets by applying an electric field based on the detection of first fluorescent moiety.

118. The method of claim 116, wherein the detection region is upstream of the merger junction.

119. The method of claim 116 or 117, wherein the one or more droplets of the plurality of droplets pair with the one or more droplets of the plurality of reagent droplets in a region in the microfluidic device that is upstream of the merger junction.

120. A method of selectively combining two or more populations of cells comprising:
flowing an emulsion comprising a first plurality of droplets comprising a first population of cells comprising at least one subpopulation of target cells through a microfluidic device, wherein each cell of the at least one subpopulation of target cells is labeled with a first fluorescent moiety;
flowing an emulsion comprising a second plurality of droplets comprising a second population of cells through the microfluidic device;
detecting a property of one or more droplets of the first plurality of droplets; and
selectively merging one or more droplets of the first plurality of droplets with one or more droplets of the second plurality of droplets based on the detection of the property.

121. The method of claim 119, wherein the property is an optical property.

122. The method of claim 120, wherein the single cell is labelled with a first fluorescent moiety and the optical property is fluorescence of the first fluorescent moiety.

123. The method of claim 120, wherein the optical property is absorbance.

124. The method of claim 119, wherein the property is size.

125. The method of claim 119, wherein the property is conductivity.

126. The method of any one of claims 119-124, wherein the selective merging comprises applying an electric field to selectively merge the one or more droplets with one or more droplets of the plurality of reagent droplets.

127. The method of any one of claims 119-124, wherein the selective merging comprises stream merging of the one or more droplets with one or more droplets of the plurality of reagent droplets.

128. The method of any one of claims 119-124, wherein the selective merging comprises pico-injection.

129. The method of any one of claims 119-127, wherein the populations of cells are populations of microbial cells and wherein the at least one subpopulation of target cells comprises a microbial cell that produces an antibiotic.

130. A method of selectively combining one or more populations of cells with one or more populations of microbes comprising:
flowing an emulsion comprising a first plurality of droplets comprising a first population of cells comprising at least one subpopulation of target cells through a microfluidic device, wherein each cell of the at least one subpopulation of target cells is labeled with a first fluorescent moiety;
flowing an emulsion comprising a second plurality of droplets comprising one or more populations of microbes through the microfluidic device;
detecting a property of one or more droplets of the first plurality of droplets; and
selectively merging one or more droplets of the first plurality of droplets with one or more droplets of the second plurality of droplets based on the detection of the property.

131. The method of claim 129, wherein the property is an optical property.

132. The method of claim 130, wherein the single cell is labelled with a first fluorescent moiety and the optical property is fluorescence of the first fluorescent moiety.

133. The method of claim 130, wherein the optical property is absorbance.

134. The method of claim 129, wherein the property is size.

135. The method of claim 129, wherein the property is conductivity.

136. The method of any one of claims 129-134, wherein the selective merging comprises applying an electric field to selectively merge the one or more droplets with one or more droplets of the plurality of reagent droplets.

137. The method of any one of claims 129-134, wherein the selective merging comprises stream merging of the one or more droplets with one or more droplets of the plurality of reagent droplets.

138. The method of any one of claims 129-134, wherein the selective merging comprises pico-injection.

139. The method of any one of claims 129-137, wherein the at least one subpopulation of target cells is labeled with a first fluorescent moiety was previously infected with a microbe that is distinct from the one or more populations of microbes in the second plurality of droplets.

140. The method of any one of claims 129-138, wherein the microbe is a bacterium.

141. The method of claim 139, wherein the microbe is a fungus.

142. The method of claim 139, wherein the microbe is a virus.

143. The method of claim 141, wherein the virus is a viral vector.

144. The method of claim 142, wherein the viral vector further comprises a clustered regularly interspaced short palindromic repeats (CRISPR) system.

145. The method of claim 142, wherein the viral vector further comprises a zinc finger nuclease (ZFN) system.

146. The method of claim 142, wherein the viral vector further comprises a transcription activator-like effector nuclease (TALEN) system.

147. A method of selectively combining one or more populations of cells with one or more small molecules comprising:
flowing an emulsion comprising a first plurality of droplets comprising a first population of cells comprising at least one subpopulation of target cells through a microfluidic device;
flowing an emulsion comprising a second plurality of droplets comprising one or more small molecules through the microfluidic device;
detecting a property of one or more droplets of the first plurality of droplets; and
selectively merging one or more droplets of the first plurality of droplets with one or more droplets of the second plurality of droplets based on the detection of the property.

148. The method of claim 146, wherein the property is an optical property.

149. The method of claim 147, wherein the single cell is labelled with a first fluorescent moiety and the optical property is fluorescence of the first fluorescent moiety.

150. The method of claim 147, wherein the optical property is absorbance.

151. The method of claim 146, wherein the property is size.

152. The method of claim 146, wherein the property is conductivity.

153. The method of any one of claims 146-151, wherein the selective merging comprises applying an electric field to selectively merge the one or more droplets with one or more droplets of the plurality of reagent droplets.

154. The method of any one of claims 146-151, wherein the selective merging comprises stream merging of the one or more droplets with one or more droplets of the plurality of reagent droplets.

155. The method any one of claims 146-151, wherein the selective merging comprises pico-injection.

156. The method of any one of claims 146-154, wherein the subpopulation of target cells labeled with a first fluorescent moiety is a rare cell subpopulation.

157. The method of any one of claims 146-155, wherein the rare cell subpopulation is a cancer cell subpopulation.

158. The method of any one of claims 119-156, wherein at least one droplet of the first plurality of droplets comprises a non-target molecule labeled with a second fluorescent moiety that is distinct from the first fluorescent moiety.

159. The method of claim 157, wherein the at least one droplet of the first plurality of droplets comprising the non-target molecule labeled with a second fluorescent moiety is not merged based on the detection of the second fluorescent moiety.

160. The method of any one of claims 119-156, wherein at least one droplet of the first plurality of droplets comprises a non-target molecule that is not labeled with a fluorescent moiety.

161. The method of claim 159, wherein the at least one droplet of the first plurality of droplets comprising the non-target molecule not labeled with a fluorescent moiety is not merged.

162. The method of claim 158 or 160, wherein the unmerged droplets are removed downstream.

163. The method of claim 158 or 160, wherein the unmerged droplets are recovered.

164. The method of claim 162, wherein the recovered unmerged droplets are recycled such that the method of any one of claims 119-160 is repeated with the recovered droplets.

165. The method of claim 163, wherein the recovered droplets are continuously recycled during performance of the method.

166. The method of any one of claims 146-164, wherein applying the electric field selectively merges one or more droplets of the first plurality of droplets with one or more droplets of the second plurality of droplets based on the detection of the first fluorescent moiety.

167. The method of any one of claims 146-165, wherein the plurality of droplets is collected in one or more output containers.

168. The method of claim 166, wherein the plurality of droplets is collected in one or more output containers via one or more collection tubes comprising valves.

169. The method of claim 166 or 167, further comprising incubating the collected plurality of droplets to allow reactions to occur in the one or more of the merged plurality of droplets to produce one or more reaction products.

170. The method of claim 168, wherein the reaction comprises a chemical synthesis reaction.

171. The method of claim 168, wherein the reaction comprises a polymerase chain reaction (PCR).

172. The method of claim 168, wherein the reaction comprises a multiple displacement amplification (MDA).

173. The method of claim 168, wherein the reaction comprises a reverse transcription reaction.

174. The method of claim 168, wherein the reaction comprises a transfection reaction.

175. The method of claim 168, wherein the reaction comprises a transduction reaction.

176. The method of claim 168, wherein the reaction comprises a transformation reaction.

177. The method of any one of claims 167-175, further comprising rupturing the plurality of droplets and recovering the reaction products for analysis.

178. The method of any one of claims 119-176, wherein the method does not comprise physically sorting the first plurality of droplets via a sorter.

179. The method of any one of claims 119-176, wherein the microfluidic device comprises a sorter, and wherein the method comprises physically sorting the first plurality of droplets via the sorter.

180. The method of claim 178, wherein the sorting of the first plurality of droplets is performed simultaneously with selectively merging the one or more droplets of the first plurality of droplets with one or more droplets of the second plurality of droplets based on the detection of the first fluorescent moiety.

181. The method of claim 178 or 179, wherein the sorting comprises physical separation of the plurality of droplets.

182. The method of any one of claims 119-180, wherein the optical detector comprises an optical fiber configured to apply excitation energy to one or more droplets of the first plurality of droplets.

183. The method of claim 181, wherein the optical fiber is configured to collect a signal produced by the application of the excitation energy to one or more droplets of the first plurality of droplets.

184. The method of any one of claims 119-182, wherein the second plurality of droplets is formed upstream of the first plurality of droplets in the microfluidic device.

185. The method of claim 183, wherein the second plurality of droplets is formed in a T-junction upstream of the first plurality of droplets.

186. The method of any one of claims 119-184, wherein the optical detector comprises a detection region.

187. The method of any one of claims 119-185, wherein the microfluidic device comprises a merger junction, wherein the one or more droplets of the first plurality of droplets is selectively merged with the one or more droplets of the second plurality of droplets by applying an electric field based on the detection of the first fluorescent moiety.

188. The method of claim 186, wherein the detection region is upstream of the merger junction.

189. The method of claim 186 or 187, wherein the one or more droplets of the first plurality of droplets pair with the one or more of the second plurality of reagent droplets in a region in the microfluidic device that is upstream of the merger junction.

190. A microfluidic device comprising:
an optical detector for detecting an optical property of one or more droplets of a plurality of droplets;
an electrode; and
an automated system, wherein the automated system applies an electric field via the electrode to selectively merge the one or more droplets of a first plurality of droplets with one or more droplets of a second plurality of droplets based on the detection of the optical property.

191. The device of claim 189, further comprising one or more droplet makers and one or more flow channels, wherein the one or more flow channels are fluidically connected to the one or more droplet makers and configured to receive one or more droplets therefrom.

192. The device of claim 190, wherein the second plurality of droplets is formed upstream of the one or more droplets of the first plurality of droplets in the one or more flow channels.

193. The device of claim 191, wherein the second plurality of droplets is formed in a T-junction upstream of the one or more droplets of the first plurality of droplets in the one or more flow channels.

194. The device of any one of claims 189-192, wherein the optical detector comprises a detection region.

195. The device of any one of claims 190-193, wherein the one or more flow channels comprise a merger junction, wherein the one or more droplets of the first plurality of droplets is selectively merged with the one or more droplets of the second plurality of droplets by activating the electrode based on the detection of an optical property.

196. The device of claim 194, wherein the detection region is upstream of the merger junction.

197. The device of any one of claims 189-195, further comprising one or more output containers wherein the plurality of droplets is collected.

198. The device of claim 196, wherein the one or more output containers are fluidically connected to one or more collection tubes comprising valves.

199. The device of any one of claims 189-197, wherein the microfluidic device does not comprise a sorter to physically sort the first plurality of droplets.

200. The device of any one of claims 189-197, wherein the microfluidic device further comprises a sorter to physically sort the first plurality of droplets.

201. The device of claim 199, wherein the sorter physically separates the first plurality of droplets.

202. A system comprising:
the microfluidic device of any one of claims 189 to 200;
a power source; and
a controller, wherein the controller is configured to selectively enable or disable an electrical connection between the power source and the electrode thereby providing an active or inactive electrode respectively.

203. A kit comprising one or more of the microfluidic devices of any one of claims 189 to 200 and the system of claim 201.

204. The kit of claim 202, further comprising instructions to carry out the methods of any one of claims 1 to 188.
